(19) United States
(12) Patent Application Publication
(10) Pub. No.: US 2017/0130229 A1
(43) Pub. Date: May 11, 2017

(54) METHODS AND COMPOSITIONS FOR INHIBITING INFECTION BY INFLUENZA AND VIRUSES

(71) Applicant: The Regents of the University of California, Oakland, CA (US)

(72) Inventors: Shou-Wei Ding, Riverside, CA (US); Yang Yang Li, Riverside, CA (US)

(21) Appl. No.: 15/317,535

(22) PCT Filed: Jun. 18, 2015
(86) PCT No.: PCT/US15/36381

§ 371 Date: Dec. 9, 2016

Related U.S. Application Data
(60) Provisional application No. 62/013,763, filed on Jun. 18, 2014.

(51) Int. Cl. C12N 15/113

(52) U.S. Cl. CPC C12N 15/1135; C12N 2310/141

(57) ABSTRACT

The disclosure provides methods and compositions for inhibiting or treating influenza comprising an miRNA that promotes Bcl-2 inhibition or mTOR/PI3K activation. The disclosure provides miR-16 and/or miR-100 nucleic acids and mimetics for the treatment or inhibition of influenza infection.

### CROSS REFERENCE TO RELATED APPLICATIONS

This application claims priority under 35 U.S.C. §119 from Provisional Application Ser. No. 62/013,763, filed Jun. 18, 2014, the disclosure of which is incorporated herein by reference.

### STATEMENT AS TO FEDERALLY SPONSORED RESEARCH

The U.S. Government has certain rights in this invention pursuant to Grant Nos. AI052447 and RC1 GM091896 awarded by the National Institutes of Health.

### TECHNICAL FIELD

The disclosure provides methods and compositions for treating and preventing viral infection.

### BACKGROUND

Influenza A virus causes seasonal infections and periodic pandemics in humans and is a major public health concern.

### SUMMARY

The disclosure identifies the structured precursors of human microRNAs 16 and 100 as the endogenous targets of NS1 and demonstrate depletion of pre-miR-100 and nuclear retention of pre-miR-16 by NS1 under physiological conditions. The disclosure also demonstrates that NS1 expression induces nuclear translocation of key components of human RNA-induced silencing complex (RISC) such as Argonautes. NS1-mediated derepression of Bcl-2 by blocking miR-16 function provides a mechanistic insight into NS1 suppression of apoptosis in infected human cells. These data together reveal that an IFN-independent host defense mediated by host miRNAs is suppressed by NS1 and this activity of NS1 plays a previously unrecognized role in the unique pathogenesis induced by Influenza A virus in single and mixed infections. Notably, the results demonstrate that miR-16 and/or miR-100, the function of which is inhibited by NS1, can be used as a novel class of therapeutics to treat or prevent influenza.

The disclosure provides a method of treating influenza viral infections in a subject, comprising administering to said subject an agent capable of inhibiting NS1 sequestering of miR-16 and miR-100.

The disclosure also provides a method of treating an influenza infection comprising inhibiting mTOR/PI3K pathway activation. In one embodiment, the inhibiting is by promoting expression or the amount of an RNAi molecule comprising an miR-100 sequence in a cell or subject. In another embodiment, the RNAi molecule comprising an miR-100 sequence is delivered to the cell or subject. In a further embodiment, the delivery is by intranasal or intraocular administration to a subject. In yet further embodiments of any of the foregoing the RNAi molecule comprising an miR-100 sequence is delivered in a pri-miR-100 form.

The disclosure provides a method of treating an influenza infection comprising inhibiting Bcl-2 activation or expression. In one embodiment, the inhibiting is by promoting expression or the amount of an RNAi molecule comprising an miR-16 sequence in a cell or subject. In another embodiment, the RNAi molecule comprising an miR-16 sequence is delivered to the cell or subject. In a further embodiment, the delivery is by intranasal or intraocular administration to a subject. In yet further embodiments of any of the foregoing the RNAi molecule comprising an miR-16 sequence is delivered in a pri-miR-16 form.

The disclosure provides a method for treating an influenza viral infection comprising administering a composition comprising (i) an RNAi comprising a miR-100 sequence; (ii) an RNAi comprising a miR-16 sequence, (iii) an NS1 protein inhibitor or (iv) any combination of (i), (ii), and (iii).

The disclosure also provides a composition for the treatment of Influenza Virus infection in a subject, comprising an agent capable of inhibiting the biological function of NS1 and (i) an RNAi comprising a miR-100 sequence; (ii) an RNAi comprising a miR-16 sequence, or (iii) a combination of (i) and (ii).

The disclosure provides an antiviral compound capable of modulating or mimicking the expression, function and/or activity of one or more host cell miRNA molecules, for treating viral infections, diseases and/or conditions comprising an miR-100 or miR-16 nucleic acid sequence.

The disclosure provides a method of treating a subject suffering from influenza comprising administering a pharmaceutically effective amount of (i) an RNAi comprising a miR-100 sequence; (ii) an RNAi comprising a miR-16 sequence, (iii) an NS1 protein inhibitor or (iv) any combination of (i), (ii), and (iii). In a further embodiment, the RNAi comprises modified nucleotide bases.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A-C shows Influenza A virus NS1 inhibits the biogenesis of miR-16 and miR-100 downstream of pri-miRNA transcription. Total RNA isolated from A549 cells 12 hours after inoculation with buffer (mock), WSN, WSNΔNS1 (ΔNS1) or Sendai virus was analyzed for the accumulation of mature miRNAs (A) and pre-miRNAs (B) by Northern blot hybridization or of pri-miRNAs and mRNAs of RIG-I, IFNβ and β-actin by semi-quantitative RT-PCR and ethidium bromide staining (C). Included for the analysis in (A) and (B) was total RNA pulled down with the Strep-tagged NS1 from 293T cells infected by the recombinant Influenza A virus (Strep rPR8, lane 5). U6 snRNA serves as a control.

FIG. 2A-E shows direct interactions of NS1 with pre-miR-16 and pri-miR-100 in infected cells. (A) WSN-infected 293T or A549 cells were treated with UV (+) and total proteins extracted for Western blot detection of NS1 (lower panel) and NS1-RNA complexes (upper panel). (B) Northern blot detection of pre-miR-16 and U6 snRNA co-immunoprecipitated by NS1 antibodies from lysates of 293T cells transfected with pCMV-miR-16 and subsequently inoculated with buffer (mock), WSN, WSNΔNS1 (ΔNS1). 10% of total lysates from WSN-infected cells was loaded as control (input). (C, D) Northern blot detection of pre-miRNAs and U6 (C) or pri-miRNAs (D) pulled down together with NS1 in 293T cells infected with the strep-tagged rPR8 virus. Total RNA from cells transfected with pCMV-miR-16 or miR-100 was loaded at left to show the position of U6, pre-miR-16 or pre-miR-100 (marker). (E) Total RNA pulled down with NS1 as in C/D was end-labeled with ³²P or biotinylated cytidine (bis)phosphate for fractionation by 15% and 6% denaturing polyacrylamide gel electrophoresis (PAGE), respectively. Molecular weight standards of proteins in kD (A) or RNAs in nucleotides (E) are shown.

FIG. 3A-B shows nuclear retention of pre-miR-16 by NS1 in infected cells. Northern blot detection of pre- and mature miRNAs from total (A), nuclear and cytoplasmic (B) extracts isolated from 293T cells transfected with a miRNA expression plasmid and subsequently infected with WSN or WSNΔNS1. RNAs extracted from cells without plasmid transfection (mock) or virus infection (−) were also analyzed as controls. To ensure successful subcellular fractionation, the same filters were also probed for a mitochondria (mito) tRNA and U6 RNA localized in the cytoplasm and nucleus, respectively.

FIG. 4 shows NS1 induces nuclear translocation of key components of human RISC in infected cells. Western blot detection of Ago1, Ago2, Dicer, PACT, TRBP and β-actin in total, nuclear and cytoplasmic extracts isolated from 293T cells 12 hours after inoculation with buffer (mock), WSN, WSNΔNS1 (ΔNS1) or Sendai virus.

FIG. 5 shows depletion of pre-miR-100, but not pre-miR-21, in A549 cells 12 and 24 hours after inoculation with Influenza virus A/WSN/33 (WSN). In contrast, mock inoculation or infection with WSNΔNS1 (ΔNS1) or Sendai virus had no inhibitory effect. Total RNA extracted from inoculated cells was analyzed by Northern blot hybridization. 18S rRNA serves as a loading control.

FIG. 6 shows predicted secondary structures of U6 small nucleolar RNA (SEQ ID NO:41) and pre-miR-16 (SEQ ID NO:2) and the limited sequence similarity shared by the two RNA molecules. miR-16-5p sequence was shown “boxed” and miR-16-3p underlined.

FIG. 7A-D shows properties of mouse vsiRNAs. (A and B) Size distribution and abundance (per million of total mature miRNAs) of virus-derived small RNAs (vsRNAs) sequenced from suckling mice infected with NoVΔB2 (A) or NoV (B) at 3 dpi, either with or without co-immunoprecipitation (IP) by antibodies specific to mouse AGOs or B2. The 5′ terminal nucleotide of vsRNAs is indicated by color. The B2-bound vsRNAs were shown as reads per million of total reads. (C) Strong enrichment of B2-bound 22-nt vsRNAs for pairs of canonical vsiRNAs with 2-nt 3′ overhangs (−2 peak) visualized by computing total pairs of 22-nt vsRNAs with different length of base-pairing. (D) A time course analysis of the accumulation of NoVΔB2 (measured by RT-qPCR of the viral genomic RNA1 normalized to β-actin mRNA) and of vsiRNAs (shown as % of the total mature miRNAs in each library) in the infected mice. 5′U % of vsiRNAs for each library was given and the virus titer at 3 dpi set as 100%.

FIG. 8A-H shows production of vsiRNAs in human 293T cells. (A-F) Size distribution and abundance (per million of total mature miRNAs) of total virus-derived small RNAs (vsRNAs) sequenced from cells 24 hours after infection with ΔNS1 mutants of PR8 and WSN strains without (input) or with co-immunoprecipitation by anti-pan AGO antibodies (AGO-IP). Strong enrichment of total and AGO-bound 22-nt RNAs of both PR8-ΔNS1 and WSN-ΔNS1 for pairs (−2 peak) of canonical vsiRNAs with 2-nt 3′ overhangs visualized by computing total pairs of 22-nt vsRNAs with different length of base-pairing (9). The 5′ terminal nucleotide of vsRNAs is indicated by color. (G and H) Read sequences along the 3′-terminal 100 nt of IAVΔNS1 mutant genome segments PB2 and NS (SEQ ID NO:6-15). Read counts (in brackets), read length, nonsequenced reads, genomic position and % of the total reads mapped to the region are indicated. The RNAs complementary to the positive/negative-strand vsiRNAs marked by a star were used as the probes subsequently to detect the influenza vsiRNAs.

FIG. 9A-D shows hDicer- and dDicer2-mediated production of influenza vsiRNAs. (A) Northern detection of the positive(+)/negative(−)-strand vsiRNAs in the parental and hDicer-KO 293T cells 24 hours after infection with the wild type (WT) or ΔNS1 viruses of PR8 strain. (B) Production of the influenza vsiRNAs in PR8-ΔNS1-infected hDicer-KO 293T cells ectopically expressing hDicer (hDcr), dDicer-2 (dDcr2), dDcr1+Loqs-PB (PB), dDcr2+Loqs-PD (PD), or dDcr2+Loqs-PD+R2D2 as indicated. The same set of RNA and protein samples extracted 24 hours after infection were used for Northern or Western blot detection of (+)/(−)-strand influenza vsiRNAs (RNA probe sequences marked by a star in FIG. 2G), precursor (pre) miR-92a, miR-92a, miR10a, U6 RNA, hDicer, the tagged Drosophila proteins, IAV nucleoprotein (NP) and NS1, or β-actin. (C and D) Size distribution and abundance of total virus-derived small RNAs per million of total miRNAs (C), and distribution of 22- and 21-nt vsiRNAs along IAV genome segment PB2 (D) from PR8-ΔNS1-infected hDicer-KO 293T cells expressing hDicer and dDicer2+Loqs-PD+R2D2, respectively. The 5′ terminal nucleotide of vsiRNAs is indicated by color.

FIG. 10A-C shows suppression of human vsiRNA biogenesis by influenza and Ebola VSRs. (A) Production of the influenza vsiRNAs in PR8-ΔNS1-infected hDicer-KO 293T cells ectopically expressing hDicer either with NS1 of PR8 or WSN, or with FLAG-tagged VP35 of Ebola virus (EBOV), Marburg virus (MARV) or bat as indicated. (B) Production of human vsiRNAs in Vero and A549 cells after PR8-ΔNS1 infection. The same set of RNA and protein samples extracted 24 hours after infection were used for Northern or Western blot detection of the influenza vsiRNAs, U6 RNA, IAV nucleoprotein (NP) and NS1, tagged VP35 variants, hDicer or β-actin. (C) Size distribution and abundance of total virus-derived small RNAs per million of total miRNAs from A549 cells infected with PR8-ΔNS1 (top), and enrichment of 22-nt canonical vsiRNA pairs (−2 peak) with 2-nt 3′ overhangs (bottom). The 5′ terminal nucleotide of vsiRNAs is indicated by color.

### DETAILED DESCRIPTION

As used herein and in the appended claims, the singular forms “a,” “and,” and “the” include plural referents unless the context clearly dictates otherwise. Thus, for example, reference to “an miRNA” includes a plurality of such miRNAs and reference to “the virus” includes reference to one or more viruses, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

It will be recognized by one of skill in the art that RNA comprises “U” (uracil) rather than “T” (thymine) and that depending upon the use of a particular molecule (e.g., vector, delivery etc.) thymidine can be replace with uracil and vice-a-versa in the sequences set forth herein.

The disclosure generally provides methods and composition for treating viral infections and more specifically influenza viral infections. The methods and compositions comprise RNAi molecules alone, combined or with other agents that inhibit viral proteins that silence natural anti-viral mechanisms.

Influenza A virus is a major cause of respiratory disease in humans and continuously re-emerges from animal reservoirs to cause pandemics in humans. Influenza viruses contain a segmented negative-strand RNA genome coding for up to 11 proteins. Unlike most RNA viruses, influenza viruses replicate exclusively in the nucleus and four of the viral proteins have been shown to participate in evading or suppressing the host innate immune responses. The non-structural protein 1 (NS1) of influenza viruses is perhaps among the best characterized viral suppressors of the host interferon (IFN) response. The IFN antagonistic activity of NS1 is related to its ability to bind and inactivate multiple host protein targets such as TRIM25 and PKR required for activation of the IFN response.

The NS1 protein has been identified and sequenced in influenza viruses and exemplary sequences can be found in the NCBI database. The NS1 proteins from influenza A, B and C do not in general show antigenic cross reactivity. Within a type (e.g., influenza A), there is considerable variation in sequence between subtypes, but some antigenic cross-reactivity depending on which antibody is used. The GenBank accession numbers of some exemplary NS1 sequences from influenza type A, subtypes H1N1, H3N2 and H5N1 respectively, are CY003340, CY003324, and DQ266101 (the contents of which are incorporated herein by reference). The GenBank accession numbers of some exemplary NS1 sequences from influenza type B are AAA43690 and BAD29872 (the contents of which are incorporated herein by reference). The NS1 protein in other strains of influenza either influenza type A, type B or type C, means a protein having the greatest sequence similarity to one of the proteins identified as an NS1 protein in known influenza strains of the same subtype, using as sequence for example, one of the GenBank accession numbers given above.

NS1 protein is a 230-amino acid protein that contains two major domains and forms a homodimer (Knipe and Howley, 2001). The amino terminal region of NS1 protein (residues 1-73) encompasses an RNA-binding domain that is able to interact non-specifically with dsRNA (Knipe and Howley, 2001). Structural and biochemical studies have shown that arginine-38 (R38) is required for binding dsRNA. This interaction is of low affinity compared to other RNA binding proteins; nevertheless, recent studies of mutant influenza viruses with impaired dsRNA binding ability have demonstrated that this function contributes to virulence. Mutations of NS1 protein that abrogate dsRNA binding resulted in attenuated viruses that grow to lower titers, induced increased IFN production, and failed to effectively block antiviral effector functions (Donelan et al., 2003; Min and Krug, 2006). However, abrogation of RNA binding attenuates virulence less than complete loss of the NS1 protein. Thus, additional sequences of the NS1 protein are also critical for virulence. A region within the amino terminal domain of NS1 protein, from amino acids 19 to 38, is required for NS1 protein-mediated inhibition of mRNA nuclear export (Qian et al., 1994), which is a key nuclear function of NS1 protein that inhibits expression of host antiviral genes. In fact, the amino terminal domain of NS1 protein is involved in its interaction with the mRNA export machinery, namely the NXF1-p15 heterodimer, Rae1 and E1B-AP5 (Satterly et al., 2007), which are mRNA export factors known to form a complex and to mediate nuclear exit of mRNAs (Bachi et al., 2000; Blevins et al., 2003; Satterly et al., 2007).

The carboxyl terminal domain of NS1 protein, amino acids 134 to 161, is also required for the inhibitory effect of NS1 protein on mRNA nuclear export (Qian et al., 1994). The carboxy terminus of NS1 protein is also termed the effector domain and is the region that binds the human 30 kD subunit of the cleavage and polyadenylation specificity factor (CPSF) and the poly(A)-binding protein II (PABII), which are involved in binding the AAUAAA polyadenylation signal and in the elongation of the poly(A) chain, respectively (Chen et al., 1999; Nemeroff et al., 1998). The interaction of NS1 protein with these proteins inhibits 3′ end processing of host mRNAs and contributes to nuclear retention of host mRNAs. A mutant influenza virus that expresses an NS1 protein with a mutated CPSF binding site is highly attenuated and cells infected with this virus produce high levels of IFN mRNA (Noah et al., 2003; Twu et al., 2006). These effects are also likely caused by changes in interactions between the mutant NS1 protein and additional host proteins directly involved in nuclear export of mRNAs. mRNA processing and export are connected—some proteins remain bound to mRNAs throughout these processes and others are exchanged with factors specific for each step. In fact, combinatorial assembly of complexes that share some common factors are being revealed as mechanisms to generate specific functions and/or redundancy (Rochette-Egly, 2005).

MicroRNAs (miRNAs) are an abundant class of short endogenous/natural RNAs that act as post-transcriptional regulators of gene expression. MicroRNAs (miRNAs) are small RNAs (˜22 nt) that regulate eukaryotic gene expression by binding to specific messenger RNA transcripts, causing the mRNAs to be degraded or causing their translation to be repressed. They are processed from longer (ca 70-80 nt) hairpin-like precursors termed pre-miRNAs by the RNAse III enzyme Dicer. MicroRNAs assemble in ribonucleoprotein complexes termed miRNPs and recognize their target sites by antisense complementarity thereby mediating down-regulation of their target genes. Near-perfect or perfect complementarity between the miRNA and its target site results in target mRNA cleavage, whereas limited complementarity between the microRNA and the target site results in translational inhibition of the target gene.

The term “miRNA” is used according to its ordinary and plain meaning and refers to a microRNA molecule found in eukaryotes that is involved in RNA-based gene regulation (see, e.g., Carrington et al., 2003, which is hereby incorporated by reference). The term can be used to refer to the single-stranded RNA molecule processed from a precursor or in certain instances the precursor itself. miRNA encompasses single-stranded RNA molecules which regulate gene expression. miRNA molecules may be between 10 and 50 nucleotides in length, preferably 15-40, more preferably 16-30 and even more preferably 17-25 nucleotides in length. Typically, miRNA molecules may be between 19 and 26 nucleotides in length. MicroRNA molecules are generally 21 to 22 nucleotides in length, though lengths of 19 and up to 23 nucleotides have been reported. The miRNAs are each processed from a longer precursor RNA molecule (“precursor miRNA” or “pre-miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) becomes part of a large complex to down-regulate a particular target gene or its gene product. MicroRNAs are encoded in the genomes of animals, plants and viruses; these genes are transcribed by RNA polymerase II as part of larger fold-back transcripts (primary miRNAs), which are processed in the nucleus by Drosha family members to form short stem-loops (pre-miRNAs), and then exported to the cytoplasm for processing by a Dicer family member to form the mature miRNA. Examples of animal miRNAs include those that imperfectly base pair with the target, which halts translation (Olsen et al., 1999; Seggerson et al., 2002).

It is estimated that miRNAs comprise 1% of genes in animals and may target up to 30% of genes in humans. A given microRNA can potentially target hundreds of genes and by modulating a whole network of gene targets, can exert dramatic effects on various cellular processes. The mechanism of microRNA function (generally down-regulation of host proteins) is distinct, but perhaps complementary, to other regulatory molecules (e.g. transcription factors). MicroRNAs have been shown to play key roles in cellular proliferation, differentiation, development and neuronal function; specific miRNAs also play a role in cancer formation, cardiovascular and metabolic diseases, and, more recently, viral infection.

Host cell miRNA modulating compounds may replicate or mimic the sequence of a host cell miRNA molecule—such compounds are referred to hereinafter as “mimic” miRNA molecules. It will be recognized the siRNA is a non-naturally occurring sequence that is substantially identical to miRNA. Mimic miRNA molecules may be exploited as a means of increasing or upregulating the expression, activity and/or function of a particular host cell miRNA. By way of example, the cell may be contacted or transfected with an miRNA molecule which mimics a host cell miRNA to be up-regulated or over-expressed. In this way, the normal miRNA expression profile of the host cell is supplemented with the mimic miRNA molecule. In one embodiment, the mimic miRNA molecules comprise nucleic acid (DNA or RNA) and may themselves be miRNA molecules.

The terms “RNA interference agent” and “RNA interference” as used herein are intended to encompass those forms of gene silencing mediated by double-stranded RNA, regardless of whether the RNA interfering agent comprises an siRNA, miRNA, shRNA or other double-stranded RNA molecule. “Short interfering RNA” (siRNA), also referred to herein as “small interfering RNA” is defined as an RNA agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, typically about 15 to about 28 nucleotides, more typically about 19 to about 25 nucleotides in length, and commonly about 20, 21, 22, 23 or 24 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Typically the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are comprised of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, viral vectors, including, e.g., adenoviral vectors, AAV vectors, among them, retroviral and lentiviral vectors and expressed from, for example, the pol III U6 promoter, or another promoter (see, e.g., Stewart, et al. RNA, 9(4):493-501, 2003, incorporated by reference herein in its entirety).

An siRNA may be substantially homologous to the target gene, target sequence or genomic sequence, or a fragment thereof. As used in this context, the term “homologous” is defined as being substantially identical, sufficiently complementary, or similar to the target mRNA, RNA, or a fragment thereof, to effect RNA interference of the target. In addition to native RNA molecules, RNA suitable for inhibiting or interfering with the expression of a target sequence include RNA derivatives and analogs. Typically the siRNA is identical to its target. The siRNA typically targets only one sequence. siRNA molecules, like miRNA, are also processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

In one embodiment the siRNA has 90-100% identity to miRNA sequence of miR-16 (e.g., the mature miR-16: uagcagcacguaaauauuggcg; SEQ ID NO:1) or miR-100 (e.g., the mature miR-100: aacccguagauccgaacuugug; SEQ ID NO:4). Each of the RNA interfering agents, such as siRNAs, can be screened for potential off-target effects by, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatizes with a variety of groups. Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated. One siRNA modifications include 2′-deoxy-2′-fluorouridine or locked nucleic acid (LAN) nucleotides and RNA duplexes containing either phosphodiester or varying numbers of phosphorothioate linkages. Such modifications are known to one of ordinary skill in the art and are described, for example, in Braasch et al., Biochemistry, 42: 7967-7975, 2003. Most of the useful modifications to the siRNA molecules can be introduced using chemistries established for antisense oligonucleotide technology. Typically, the modifications involve minimal 2′-O-methyl modification, and preferably excludes such modification. Modifications also can exclude modifications of the free 5′-hydroxyl groups of the siRNA.

siRNA can be delivered by a vector, including both RNA and DNA vectors. Where DNA vectors will comprise a DNA sequence encoding/transcribed to the siRNA. When an RNA vector is used (e.g., a retroviral vector), the siRNA sequence is transcribed from the viral genome.

An “miR-16 nucleic acid sequence” or “miR-16 inhibitor” includes the full length precursor of miR-16, or complement thereof or processed (i.e., mature) sequence of miR-16 and related sequences set forth herein, as well as 5, 6, 7, 8, 9, 10, 11, 12, 13, 14, 15, 16, 17, 18, 19, 20, 21, 22, 23, 24, 25, 26, 27, 28, 29 or more nucleotides of a precursor miRNA or its processed sequence, or complement thereof, including all ranges and integers there between. In certain embodiments, the miR-16 nucleic acid sequence or miR-16 inhibitor contains the full-length processed miRNA sequence or complement thereof and is referred to as the “miR-16 full-length processed nucleic acid sequence” or “miR-16 full-length processed inhibitor sequence.” In still further aspects, the miR-16 nucleic acid comprises at least one 5, 6, 7, 8, 9, 10, 11, 12, 13, 14, 15, 16, 17, 18, 19, 20, 21, 22, 23, 24, 25, 50 nucleotide (including all ranges and integers there between) segment or complementary segment of a miR-16 that is at least 75, 80, 85, 90, 95, 98, 99 or 100% identical to SEQ ID NOs provided herein. The general term miR-16 includes all members of the miR-16 family that share at least part of a mature miR-16 sequence. In still further aspects, the miR-16 nucleic acid comprises at least 5, 6, 7, 8, 9, 10, 11, 12, 13, 14, 15, 16, 17, 18, 19, 20, 21, 22, 232, 24, 25, 50 nucleotides (including all ranges and integers there between) segment of miR-16 that is at least 75, 80, 85, 90, 95, 98, 99 or 100% identical to SEQ ID NOs:1-3.

An “miR-100 nucleic acid sequence” or “miR-100 inhibitor” includes the full length precursor of miR-100, or complement thereof or processed (i.e., mature) sequence of miR-100 and related sequences set forth herein, as well as 5, 6, 7, 8, 9, 10, 11, 12, 13, 14, 15, 16, 17, 18, 19, 20, 21, 22, 23, 24, 25, 26, 27, 28, 29 or more nucleotides of a precursor miRNA or its processed sequence, or complement thereof, including all ranges and integers there between. In certain embodiments, the miR-100 nucleic acid sequence or miR-100 inhibitor contains the full-length processed miRNA sequence or complement thereof and is referred to as the “miR-100 full-length processed nucleic acid sequence” or “miR-100 full-length processed inhibitor sequence.” In still further aspects, the miR-100 nucleic acid comprises at least one 5, 6, 7, 8, 9, 10, 11, 12, 13, 14, 15, 16, 17, 18, 19, 20, 21, 22, 23, 24, 25, 50 nucleotide (including all ranges and integers there between) segment or complementary segment of a miR-100 that is at least 75, 80, 85, 90, 95, 98, 99 or 100% identical to SEQ ID NOs provided herein. The general term miR-100 includes all members of the miR-100 family that share at least part of a mature miR-100 sequence. In still further aspects, the miR-100 nucleic acid comprises at least 5, 6, 7, 8, 9, 10, 11, 12, 13, 14, 15, 16, 17, 18, 19, 20, 21, 22, 232, 24, 25, 50 nucleotides (including all ranges and integers there between) segment of miR-100 that is at least 75, 80, 85, 90, 95, 98, 99 or 100% identical to SEQ ID NOs:4-5.

MicroRNAs are an important component of viral-host interactions and have been shown to influence the outcome of viral infections. Recent studies have demonstrated that specific host microRNAs are up- or down-regulated upon infection with a particular virus and that some of these host microRNAs have target sites against specific viruses. This has led to the suggestion that microRNAs could mediate anti-viral defense; for example, mir-32 was shown to limit the replication of primate foamy virus (PFV) in human cells by targeting regions in the PFV genome (Lecellier et al. 2005). In another study, mir-24 and mir-93 were shown to target vesicular stomatitis virus, leading to decreased replication of the virus in mice (Otsuka et al. 2007). However, rather than being “anti-viral”, the host microRNAs that target viruses may, in fact, be exploited by the viruses for persistence. From an evolutionary point of view, if the host miRNA target sites were disadvantageous to the virus, the virus could readily evolve to eliminate these sites (requiring only a single mutation) (Mahajan et al. 2008). For example, it was shown that host microRNAs (mir-28, mir-125b, mir-150, mir-223 and mir-382) down regulate HIV mRNA and may be used by the virus to avoid being eliminated by the immune system (Huang et al. 2007). Furthermore, it is known that the host microRNA, mir-122, can actually be exploited by the virus to upregulate viral genes (Jopling et al. 2006).

The work listed above demonstrates that human and mouse microRNAs can play a pro- or anti-viral function by interacting with viral sequences; however, antiviral therapies based on microRNAs that specifically interact with viral genomes possess a number of disadvantages: 1) the viruses can mutate/evolve to escape the microRNA-target interactions, and 2) the identified microRNAs would be limited to function against the virus with the target site, rather than holding broad anti-viral potential.

More recently, it has been shown that cellular microRNAs that are induced or down regulated upon viral infection can also modulate host genes, which are co-factors for viral infection. For example, the miRNA cluster mir-17/92 was shown to be decreased upon HIV-1 infection and was shown with knockdown experiments to effect HIV-1 replication; this microRNA targets histone acetylase protein PCAF, which is a co-factor for the viral Tat protein (Triboulet et al. 2007). In plant and invertebrate hosts, cytoplasmic replication of viral RNA genomes triggers specific antiviral RNAi response directed by virus-derived siRNAs so that viral suppression of RNAi is essential for infection.

Many mammalian viruses such as Ebola, La Crosse, HIV, and hepatitis C viruses encode proteins capable of suppressing RNAi, some of which bind to dsRNA in vitro but share no structural similarities to NS1. However, a physiological role for the viral RNAi suppression activity in mammalian viral infection is unclear since induction of specific antiviral RNAi response directed by virus-derived siRNAs has been shown only in plants and invertebrates.

Nuclear replication of the influenza viral RNA genome does not appear to induce biogenesis of virus-derived siRNAs or miRNAs (vsiRNAs) in the infected mammalian cells. The disclosure demonstrates that this lack of induced biogenesis of vsiRNA is due to the RNAi suppressor activity of NS1.

The disclosure demonstrates that expression of NS1 during infection interferes with the biogenesis of specific human host miRNAs by direct binding of NS1 to the structured miRNA precursors. The disclosure demonstrates that miR-16 and miR-100 are sequestered by NS1 of the influenza virus. miR-16 and miR-100 control expression of many target genes in pathways essential for virus infection and/or immune responses. In particular, miR-16 induces apoptosis by repressing the expression of Bcl-2, a known suppressor of apoptosis.

The disclosure demonstrates that suppression of the function of specific host miRNAs by NS1 under physiological conditions promotes viral infection. Accordingly, the disclosure provides a new paradigm for the action of mammalian viral suppressors of RNAi (VSRs) such as NS1.

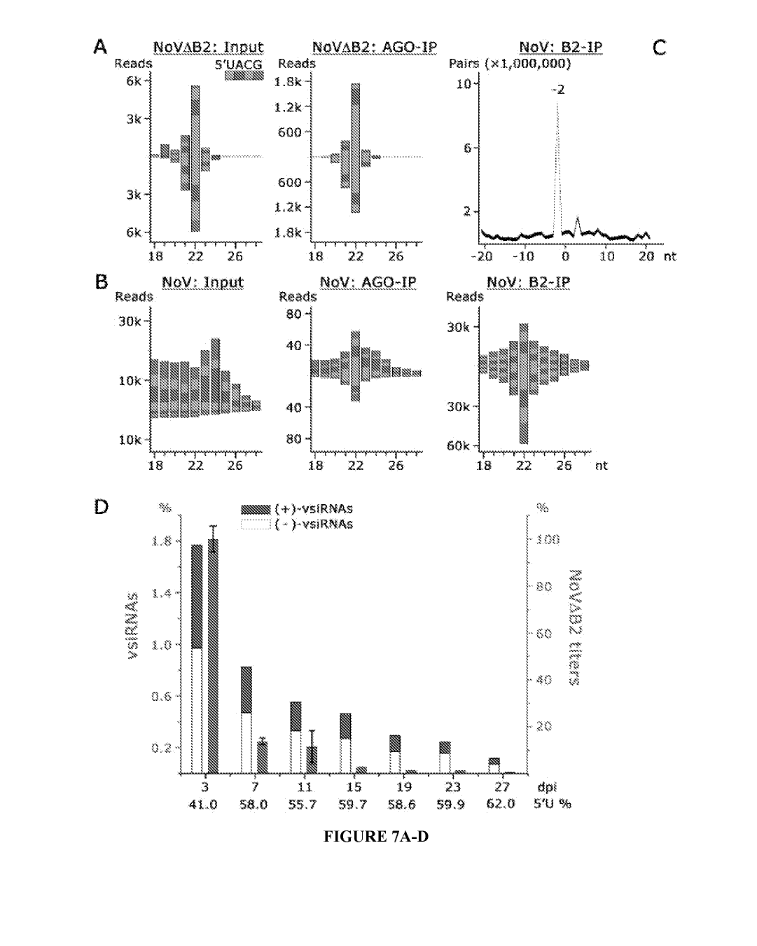

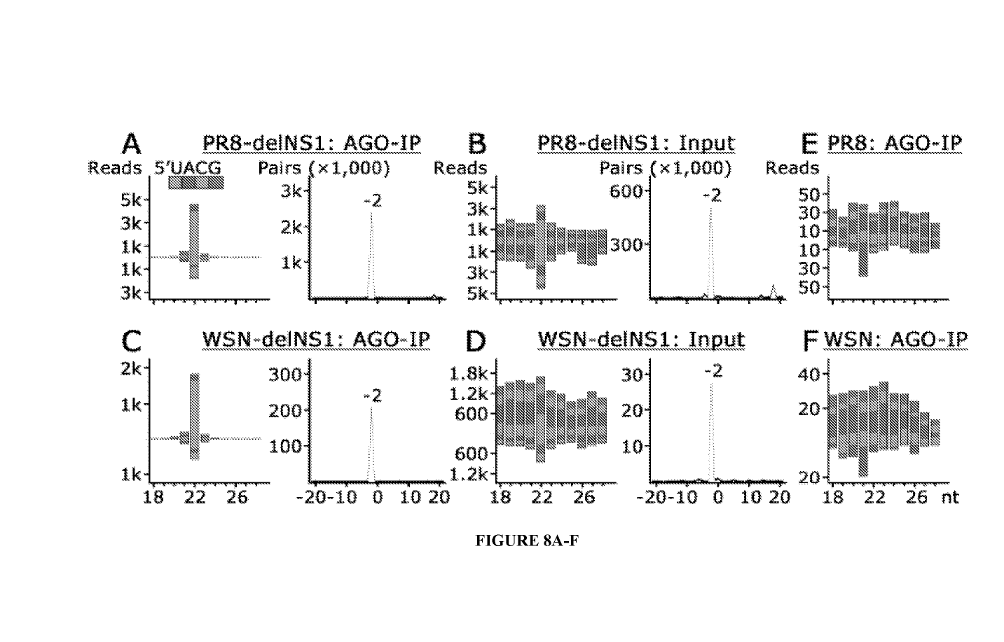

To examine the role of vsiRNA accumulation, a strategy was developed for the identification of human vsiRNAs. First, the properties of mouse vsiRNAs were defined. Suckling mice accumulate highly abundant vsiRNAs in limp muscle tissues following intraperitoneal inoculation by NoVΔB2, which contained point mutations in the viral RNA genome to prevent translation of the B2 protein. Data showed that 41% of the vsiRNAs sequenced from mice 3 days post inoculation (dpi) with NoVΔB2 contained uridine as the 5′-terminal nucleotide (5′U) (FIG. 7A). Sequencing the total small RNAs co-immunoprecipitated with mouse AGOs from the same NoVΔB2-infected mice revealed a population of vsiRNAs with 63.3% 5′U as well as the size preference and strand ratio known for vsiRNAs (FIG. 7A). A time course analysis of vsiRNAs during NoVΔB2 infection and clearance in mice showed that 5′U vsiRNAs increased to approximately 60% in total vsiRNAs sequenced from mice from 7 to 27 dpi (FIG. 7D). Therefore, mouse vsiRNAs are in vivo loaded in Argonautes and exhibit preference for both 5′U and 22-nt, similar to mammalian miRNAs.

Libraries of small RNAs co-immunoprecipitated by antibodies specific to the viral B2 protein or mouse AGOs from NoV-infected mice at 3 dpi were prepared and sequenced. NoV-derived small RNAs were highly enriched by B2 co-immunoprecipitation. In contrast to strong positive strand bias and random size distribution of the input population, B2-bound NoV small RNAs had approximately equal strand ratios for all sizes with a peak for 22-nt and no preference for 5′U (FIG. 7B). Moreover, NoV 22-nt RNAs in complex with B2 were markedly enriched for pairs with 20-nt perfect duplex and 2-nt 3′-overhangs (FIG. 7C), another property of vsiRNAs from NoVΔB2-infected mice. However, NoV small RNAs co-immunoprecipitated with AGOs were extremely low compared to NoVΔB2-infected mice (FIGS. 7A-B). Thus, in addition to dicing suppression, B2 also sequesters duplex vsiRNAs and prevents loading into AGOs, consistent with previous studies in vitro. The dual mode of B2 in RNAi suppression may explain why wild-type NoV infection is associated with low abundant vsiRNAs that escape detection by deep sequencing of small RNAs either with or without Argonaute co-immunoprecipitation.

The mouse study suggests an approach to identify mammalian vsiRNAs by sequencing the small RNAs co-immunoprecipitated with AGOs from cells infected with RNA virus mutants not expressing the cognate VSR. Accordingly, experiments were performed on human Influenza A virus (IAV) since its non-structural protein 1 (NS1) exhibits VSR activity and binds dsRNA with two positively charged antiparallel α-helices similarly to B2 proteins of NoV and Flock house virus. Since virus small RNAs sequenced from human somatic cells infected with wild-type IAV do not exhibit the properties of vsiRNAs, it has not been possible to determine if NS1 suppresses an RNAi response during IAV infection.

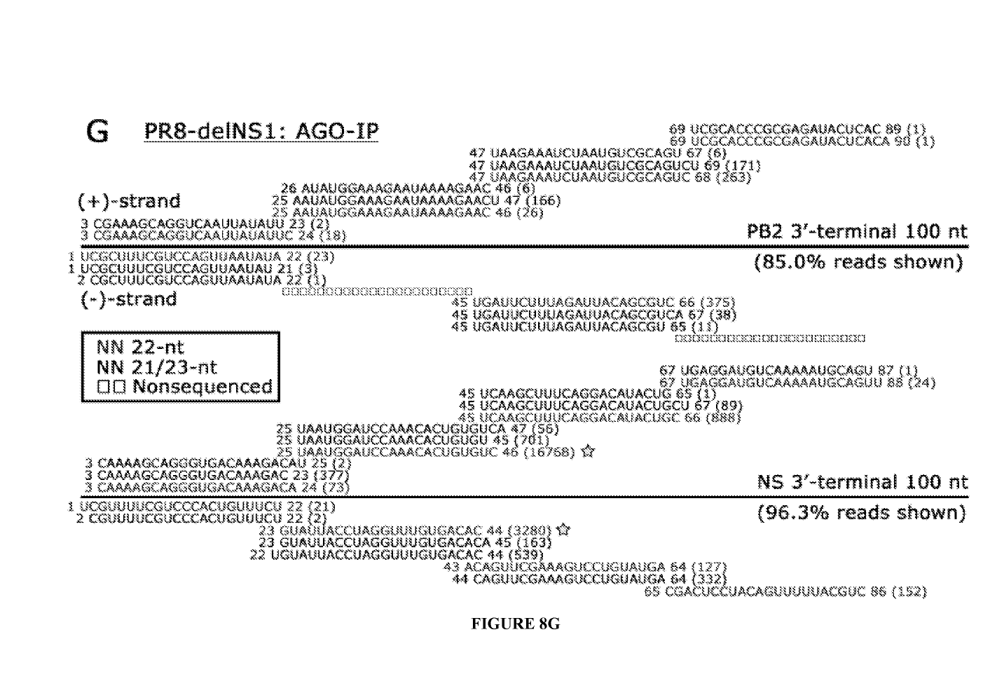

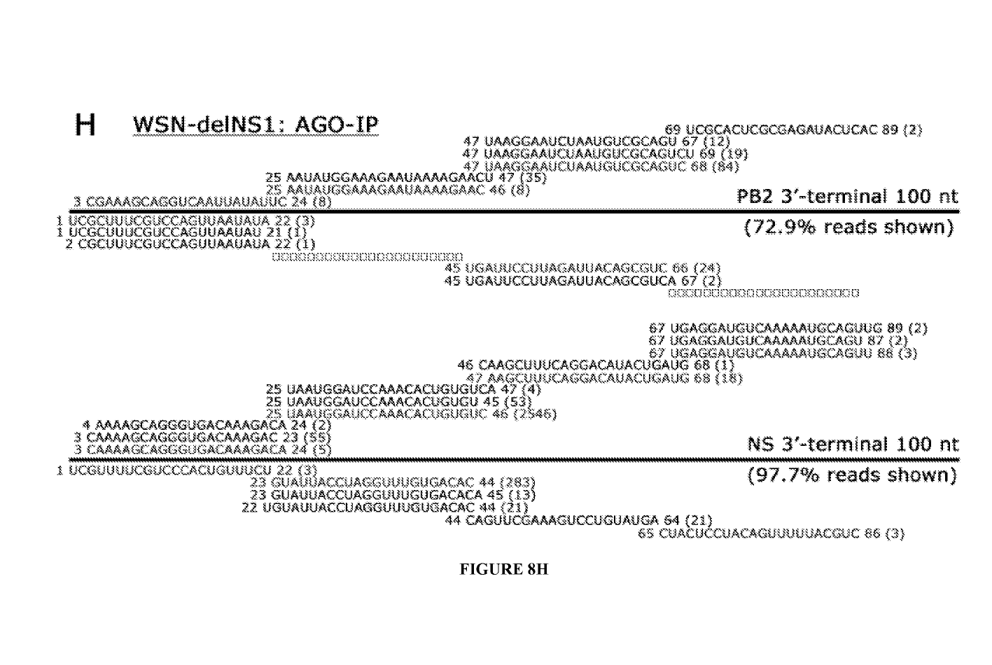

vsiRNAs were detected in a population of IAV-derived small RNAs co-immunoprecipitated with AGOs from human 293T cells infected with a mutant IAV; PR8-ΔNS1, a NS1-deletion mutant of IAV strain A/Puerto Rico/8/1934(H1N1) (FIG. 8). 93.6% of the 41,324 virus reads, cloned by a protocol dependent on the monophosphate at the 5′-end, were in the 21- to 23-nt size range of Dicer products with 22-nt as the most dominant size for both the positive and negative strands (FIG. 8). The 22-nt RNAs of IAV were highly enriched for both 5′U (71.5%) and 20-nt perfect base-paired duplexes with 2-nt 3′ overhangs (FIG. 8A). The influenza vsiRNAs were abundant, representing 0.34% of the total sequenced reads and equal to 0.81% of the total mature miRNA content in the library. Moreover, 91.8% of the virus reads were derived from the terminal 100-nt regions of the eight virion RNA segments and these terminal virus reads formed successive (or phased) complementary pairs of vsiRNAs (FIG. 8G). These findings suggest that the terminal viral dsRNA replicative intermediates served as the precursors of the influenza vsiRNAs.

An independent library of small RNAs co-immunoprecipitated with AGOs from 293T cells infected by WSN-ΔNS1, a NS1-deletion mutant of the human IAV strain A/WSN/1933(H1N1) was also sequenced. 90.9% of the AGO-bound WSN reads were 21 to 23 nt long and 88.6% mapped to the termini of the viral genome segments with successive pairs of vsiRNAs (FIGS. 8C, 8H). The dominant 22-nt reads of WSN-ΔNS1 were enriched for both 5′U (80.3%) and perfect siRNA pairs with 2-nt 3′ overhangs (FIG. 8C). These finding together show that the AGO-bound vsiRNAs detected in human 293T cells after infection with PR8-ΔNS1 and WSN-ΔNS1 strain are strikingly similar to each other and to the mouse vsiRNAs described above, providing evidence for the production of abundant vsiRNAs in human somatic cells.

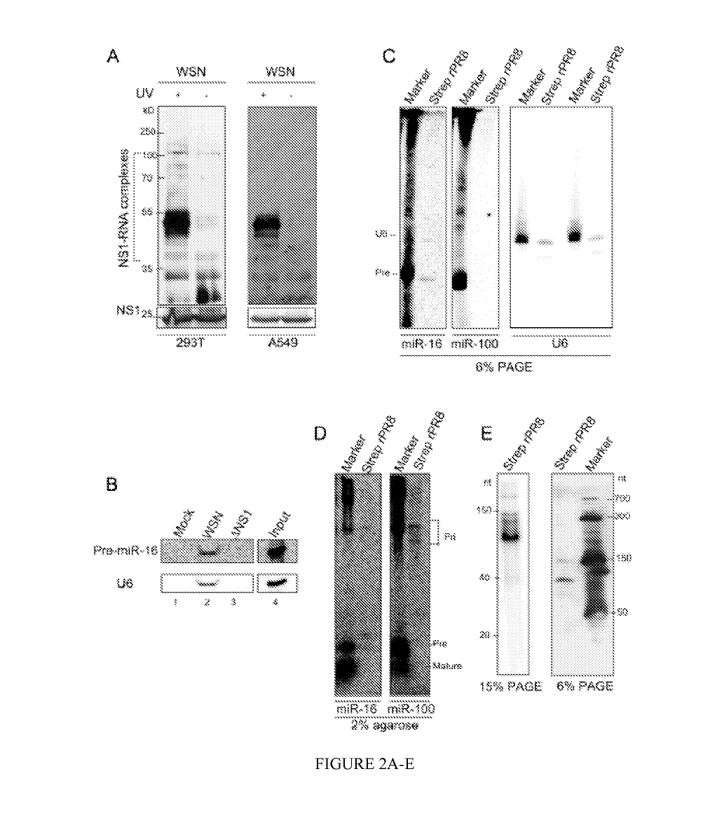

Total small RNAs from 293T cells infected with either PR8-ΔNS1 or WSN-ΔNS1 were also sequenced without Argonaute co-immunoprecipitation. The 22-nt virus reads in each library were highly enriched for canonical pairs of vsiRNAs (FIGS. 8B and 8D). In both libraries, however, the 22-nt peak was weak for the positive- and negative-strand virus reads and there were abundant virus reads outside the size range of Dicer products (FIGS. 8B and 8D). Moreover, virus reads co-immunoprecipitated with AGOs from 293T cells infected with wild-type PR8 and WSN strains were extremely low abundant without preference in size or for 5′U (FIGS. 2E/2F). These findings provide further validation for the strategy to identify mammalian vsiRNAs by combining VSR ablation with Argonaute co-immunoprecipitation.

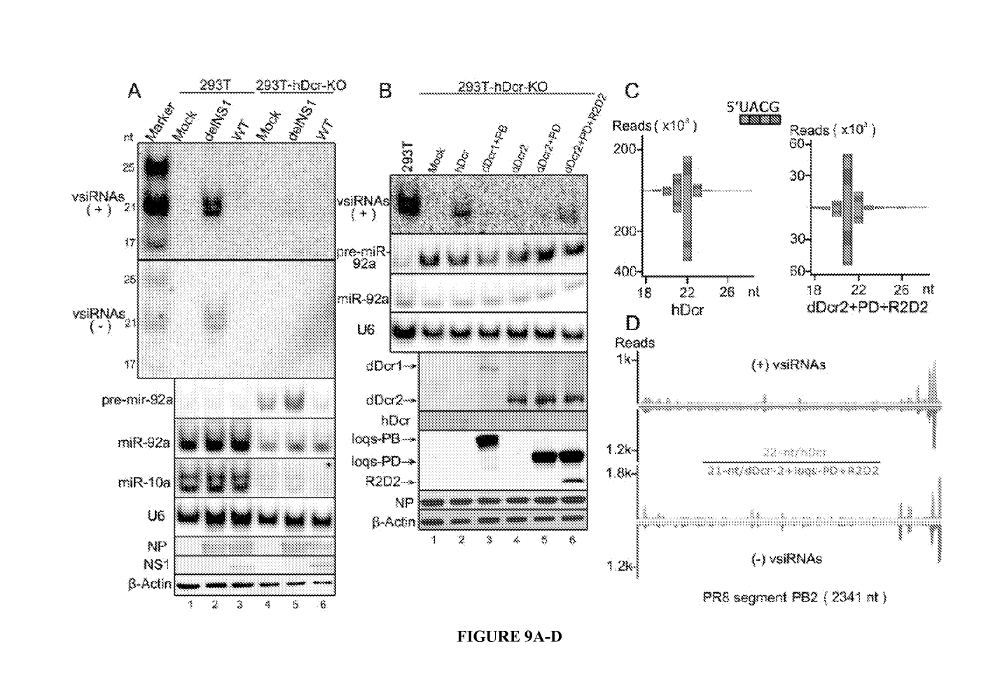

Both the positive- and negative-strand influenza vsiRNAs were readily detectable as discrete bands in the 21- to 22-nt size range by RNA hybridization in 293T cells infected with PR8-ΔNS1 (FIG. 9A). Thus, the production, size and abundance of the influenza vsiRNAs in 293T cells as revealed by deep sequencing were verified by an independent approach. To investigate the role of human Dicer (hDicer) in the biogenesis of the influenza vsiRNAs, a human 293T cell line knockout (KO) for hDicer was used. Both the positive- and negative-strand influenza vsiRNAs became undetectable in the hDicer-KO cells unlike the parental cells (FIG. 9A). However, ectopic expression of hDicer from a co-transfected plasmid encoding an hDicer cDNA rescued vsiRNA production in the hDicer-KO cells (FIG. 9B). Together with the canonical properties of the sequenced influenza vsiRNAs (FIG. 8), these findings reveal a new somatic function of hDicer in the production of the influenza vsiRNAs during an authentic infection of the differentiated human cells.

hDicer shares the same domain architecture with Drosophila Dicer-2 (dDicer2), which processes virus-specific dsRNA into vsiRNAs in fruit fly somatic tissues. Co-expression of dDicer2 with its dsRNA-binding protein partners, Loquacious isoform PD (Loqs-PD) and R2D2, rescued production of the influenza vsiRNAs in hDicer-KO 293T cells (FIG. 9B). However, influenza vsiRNAs remained undetectable in the hDicer-KO cells co-expressing Loqs-PB and dDicer1 (FIG. 9B), which produces miRNAs in Drosophila and lacks the DExDc domain conserved between hDicer and dDicer2. A size shift of the vsiRNAs to predominantly 21-nt was detected by deep sequencing in the hDicer-KO cells ectopically expressing dDicer2 compared to those expressing hDicer (FIG. 9C). However, the same set of dsRNA precursors may be recognized and processed by dDicer2 and hDicer since the distribution pattern of hot spot vsiRNAs over the genomic RNAs of PR8-ΔNS1 was highly similar for the 21- and 22-nt vsiRNAs sequenced from the hDicer-KO cells expressing dDicer2 and hDicer, respectively (FIG. 9D). These findings further support a somatic function of hDicer in the biogenesis of vsiRNAs.

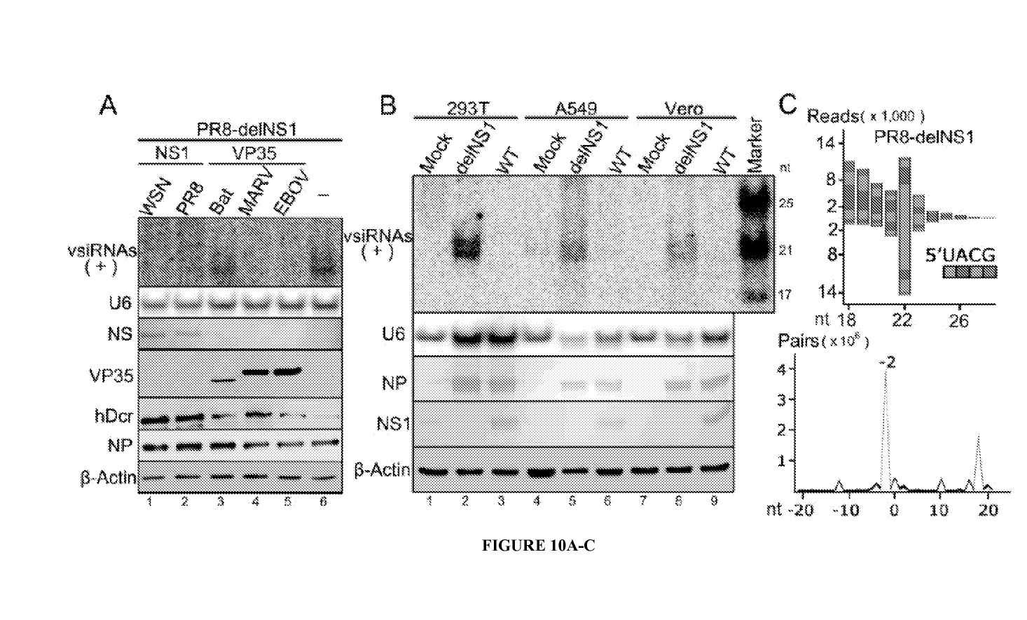

The influenza vsiRNAs were undetectable by RNA hybridization in 293T cells infected by PR8-WT, which directs expression of NS1 (FIG. 9A, lane 3). Ectopic expression of NS1 of IAV strain PR8 or WSN actively inhibited production of the influenza vsiRNAs induced by PR8-ΔNS1 infection (FIG. 10A). These findings reveal a new activity of NS1 to inhibit vsiRNA production during an authentic infection of human somatic cells, and are consistent with the lack of canonical vsiRNAs associated with wild type IAV infection shown by deep sequencing here (FIG. 8E). Moreover, strong suppression of the influenza vsiRNA biogenesis by virion protein 35 (VP35) of Ebola virus (FIG. 10A), a known VSR with dsRNA-binding activity was also noted. Production of the influenza vsiRNAs was also suppressed by ectopic expression of VP35 encoded by Marburg virus from the same Filoviridae as Ebola virus, but not by the distantly related VP35 encoded by the bat genome (FIG. 10A).

Human lung epithelial cells A549 and African green monkey epithelial cells Vero are commonly used for IAV pathogenesis studies. Production of the influenza vsiRNAs was efficiently induced in both of the somatic cell lines by infection with PR8-ΔNS1, but not PR8-WT (FIG. 10B). Deep sequencing of total small RNAs from A549 cells infected with PR8-ΔNS1 revealed a typical population of vsiRNAs with strong preference for 22-nt and 5′U, approximately equal ratios of the positive and negative strand RNAs in the 21- to 23-nt size range and enrichment of 22-nt vsiRNA pairs containing 2-nt 3′ overhangs (FIG. 10C). These findings together illustrate that production of abundant vsiRNAs is a conserved immune response to IAV infection in distinct human and monkey somatic cells.

Since hDicer is known to produce only miRNAs in somatic cells, the disclosure provides evidence that identifies a new somatic function of hDicer in the biogenesis of the highly abundant influenza vsiRNAs. The data show that dDicer-2, which produces endogenous and viral siRNAs in Drosophila, can mediate the biogenesis of the influenza vsiRNAs in place of hDicer in somatic cells. These findings demonstrate that the rodent-specific isoform of Dicer is not essential for the RNAi response to virus infection in mammals and that similar to the intracellular dsRNA sensors RIG-I and PKR, mammalian Dicer also has access to the virus dsRNA in somatic cells. These results show that the immune sensing of viral dsRNA and its subsequent processing into vsiRNAs by Dicer occur in somatic cells. Potent suppression of the vsiRNA biogenesis by mammalian VSRs NS1 and VP35 suggests that viruses from two different families have evolved to target the somatic function of hDicer by key virulence factors, providing the first evidence for a physiological activity during infection for human VSRs characterized previously only in experimental or heterologous RNAi systems.

The disclosure provides a method of identifying vsiRNAs by combining VSR ablation with deep sequencing of small RNAs co-immunoprecipitated with AGOs from infected cells. The disclosure also provide compositions and method of treating viral infections.

The disclosure also provides a method of treating an influenza infection comprising inhibiting mTOR/PI3K pathway activation. In one embodiment, the inhibiting is by promoting expression or the amount of an miR-100 in a cell or subject. In another embodiment, the miR-100 is delivered to the cell or subject. In another embodiment, the delivery is by intranasal or intraocular administration to a subject. In yet another embodiment, the miR-100 is delivered in a pri-miR-100 form.

The disclosure also provides a method of treating an influenza infection comprising inhibiting Bcl-2 activation or expression. In one embodiment, the inhibiting is by promoting expression or the amount of an miR-16 in a cell or subject. In another embodiment, the miR-16 is delivered to the cell or subject. In another embodiment, the delivery is by intranasal or intraocular administration to a subject. In yet a further embodiment, the miR-100 is delivered in a pri-miR-16 form.

The disclosure also provides a composition for the treatment of Influenza Virus infection in a subject, comprising an agent capable of inhibiting the biological function of NS1 (an NS1 protein inhibitor).

As used herein, an “NS1 protein inhibitor” is an agent that inhibits NS1 protein activity. The inhibitor can be a small molecule, peptide, polypeptide, or inhibitor nucleic acid. In one embodiment, the agent is a small molecule inhibitor (see, e.g., U.S. Pat. Publ. No. 2009/0170840A1 and 2012/0178749A1 to Roth et al., the disclosures of which are incorporated herein by reference). In another embodiment, the agent is a nucleic acid molecule that binds to and inactivates that NS1 protein by interacting with the dsRNA binding domain of NS1. Alternatively, the agent can be a peptide mimetic that binds to and inactivates the effector domain of NS1.

The disclosure also provides an antiviral compound capable of modulating or mimicking the expression, function and/or activity of one or more host cell miRNA molecules, for treating viral infections, diseases and/or conditions comprising an miR-100 or miR-16 nucleic acid sequence (see, e.g., SEQ ID NOs:1-5).

The disclosure provides a method of treating a subject suffering from influenza comprising administering a pharmaceutically effective amount of a miR-16 and/or miR-100 nucleic acid composition. The composition can comprise a vector that expresses a miR-16 and/or miR-100 or may comprise a formulation that promotes uptake of a miR-16 and/or miR-100. In any of the foregoing embodiments the nucleic acid sequence of an miR-16 or -100 can comprise modified nucleotide bases.

In another embodiment, the disclosure provides a method of treating an influenza virus infection comprising administering to a subject in need thereof a therapeutically effective amount of (i) an miR-16, an siRNA comprising a sequence that is 90-100% identical to miR-16, or a sequence that is an analog of miR-16 comprising 1 or more modified bases; (ii) an miR-100, an siRNA comprising a sequence that is 90-100% identical to miR-100, or a sequence that is an analog of miR-100 comprising 1 or more modified bases; (iii) and NS1 protein inhibitor; and (iv) any combination of (i), (ii) and (iii). In any of the foregoing, the composition of any of (i) to (iv) can be prepared in a pharmaceutically acceptable carrier suitable for a desired delivery route.

The steps involved in introducing an RNAi molecule (e.g., siRNA, miRNA etc.) into a cell are well known and may involve, for example, the use of transfection protocols or vectors (for example eukaryotic gene expression vectors) such as transcription cassettes, plasmids or viral vectors. In some embodiments, the nucleo-base can be charge neutralized by using, e.g., RNA binding proteins and a protein transduction domain and/or using a modified phosphate backbone.

Method of promoting uptake of siRNA or other RNAi molecules are known. For example, the RNAi molecules of the disclosure (e.g., miR-16, miR-100, siRNA homologs thereof and the like), can be modified or combined according to the teachings of U.S. Pat. Publ. Nos: 2009/0093425A1, 2009/0093026A1, and 2012/0142763A1; see also WO2014/031575A1, the disclosure of each of the foregoing are incorporated herein by reference). Thus, the siRNA molecules of the disclosure can be modified to promote uptake by cells.

In some embodiments, the RNA interference agent(s) (and optionally including an NS1 or other VSR inhibitor) is delivered or administered in a pharmaceutically acceptable carrier. Additional carrier agents, such as liposomes, can be added to the pharmaceutically acceptable carrier. In another embodiment, the RNA interference agent is delivered by a vector in a pharmaceutically acceptable carrier to the cells of a subject.

In some embodiments, the composition(s) (e.g., RNA interference agent compositions and/or RNAi+VSR inhibitor) used in the methods described herein are taken up actively by cells in vivo following intravenous injection, e.g., hydrodynamic injection, without the use of a vector, illustrating efficient in vivo delivery of the RNA interfering agents. One method to deliver the siRNAs is catheterization of the blood supply vessel of a target organ.

Other strategies for delivery of the RNA interference agents, e.g., the siRNAs or shRNAs used in the methods of the disclosure, may also be employed, such as, for example, delivery by a vector, e.g., a plasmid or viral vector. In one embodiment, the vector delivering the RNA interference agent is a regulatable vector, such as tetracycline inducible vector. As used herein, the term “vector” refers to a nucleic acid molecule capable of transporting another nucleic acid to which it has been linked. One type of vector is a “plasmid”, which refers to a circular double stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “recombinant expression vectors”, or more simply “expression vectors.” In general, expression vectors of utility in recombinant DNA techniques are often in the form of plasmids. However, the invention is intended to include other forms of expression vectors, such as viral vectors (e.g., replication defective retroviruses, replication competent retroviruses (e.g., MLV-based vectors), lentiviruses, adenoviruses and adeno-associated viruses), which serve equivalent functions.

Typically, transfection protocols, including reverse transfection protocols, utilize conditions rendering cell membranes permeable to compounds such as nucleic acids. By way of example, it may be possible to transfect (or reverse transfect) host miRNA mimic and/or inhibitor molecules into cells using electroporation, heat shock and/or compounds such as calcium phosphate or lipid-based reagents. In one embodiment, a PTD-DRB fusion construct can be used (see, e.g., U.S. Pat. Publ. No. 20090093026 A1, incorporated herein by reference). In another embodiment, the RNA nucleic acid itself is modified to reduce the RNA backbone's charge through charge neutralization moieties (see, e.g., U.S. Pat. Publ. No. 20090093425 A1, incorporated herein by reference).

Additionally, or alternatively, the host miRNA modulating compound may be introduced into the cell by means of a gene gun. In such cases, the nucleic acid to be introduced may be associated with or otherwise conjugated to a particle which can be delivered directly to the cell.

To identify any modulation of viral replication and/or propagation it may possible to modify the viruses to include some form of reporter element. For example, the viruses may be modified to include a fluorescent or luciferase reporter moiety. Where two or more viruses are to be added to a cell, each virus may be modified to include different reporter moieties. The expression of such moieties may easily be detected using, for example, optical plate readers and the like. In all cases the amount or number of fluorescent or luciferase moiety, plaques, hemolysis, cell lysis and/or haemagglutination detected, correlates with modulated viral propagation and/or replication.

As stated, modulated viral propagation and/or replication may easily be detected by comparing the results obtained from an infection in the absence of a mimic miRNA or other nucleic acid inhibitor that has similar activity as miR-16 and miR-100 with the results obtained from samples which include a delivery of a miR-16 and miR-100 or mimic thereof.

By over-expressing, up-regulating or mimicking the miRNA molecules provided herein, it is possible to inhibit viral propagation and/or replication in host cells. In one embodiment, a pre-miRNA is administered to a subject or host cell, which is then processed by the subject/cell to provide the mature miRNA. In another embodiment, the mature miRNA is administered to a subject or cell. Accordingly, in one embodiment, the disclosure provides a multi-species antiviral compound capable of modulating the expression, function and/or activity of one or more host cell miRNA molecules, for treating viral infections, diseases and/or conditions.

In another embodiment, the disclosure provides a multi-species antiviral compound capable of modulating the expression, function and/or activity of one or more host cell miRNA molecules for the manufacture of a medicament for treating viral infections, diseases and/or conditions.

In yet another embodiment, the disclosure provides a method of treating a subject suffering from a viral infection, disease and/or condition, said method comprising the steps of administering a pharmaceutically effective amount of a multi-species antiviral compound capable of modulating the expression of one or more host cell miRNA molecules.

In one embodiment the one or more host cell miRNA molecules are selected from pre-miRNA miR-16, mature miR-16, pre-miRNA miR-100 and mature miR-100 or non-naturally occurring siRNAs having 99% identity to any of the foregoing and analogs of any of the foregoing having modified bases.

Accordingly, one embodiment of this disclosure provides (a) multi-species antiviral compounds comprising nucleic acids selected SEQ ID NO:1, 2, 3, 4, and 5, homologs or analogs thereof for use in treating viral infections; (b) the use of such nucleic acid compounds for the manufacture of a medicament for treating viral infections; and (c) a method of treating viral infections, said method comprising the steps of administering a pharmaceutically effective amount of a composition comprising the nucleic acid compounds above.

It should be understood that the compositions, medicaments and methods provided by this disclosure may comprise or use one or more of the sequences provided above. For example, a composition or medicament for treating a viral infection may comprise two or more of the RNAi molecules described herein. Compositions, medicaments and methods which pool or combine compounds selected from those may be particularly useful. Furthermore, the compositions, medicaments and/or methods described herein may be combined with any number of existing antiviral compounds or treatments. For example, the RNAi agents above may be delivered in combination with NS1 inhibitor as set forth in U.S. Patent Publ. Nos. 20090170840 and 20070218122, the disclosure of which are incorporated herein.

The disclosure provides pharmaceutical compositions comprising any of the nucleic acid compounds described above in association with a pharmaceutically acceptable excipient, carrier or diluent. Such compositions may find application in, for example, the treatment of viral infections and/or diseases and/or conditions caused or contributed to by, viruses.

The pharmaceutical compositions provided by the disclosure are formulated as sterile pharmaceutical compositions. Suitable excipients, carriers or diluents may include, for example, water, saline, phosphate buffered saline, dextrose, glycerol, ethanol, ion exchangers, alumina, aluminium stearate, lecithin, serum proteins, such as serum albumin, buffer substances such as phosphates, glycine, sorbic acid, potassium sorbate, partial glyceride mixtures of saturated vegetable fatty acids, water salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycon, sodium carboxymethylcellulose, polyacrylates, waxes, polyethylene-polypropylene-block polymers, polyethylene glycol and wool fat and the like, or combinations thereof.

Said pharmaceutical formulation may be formulated, for example, in a form suitable for oral or parenteral administration. In one embodiment, the pharmaceutical formulation is for intranasal or intraocular delivery.

Other delivery methods include delivery of the RNAi (pre- and/or mature) agents of the disclosure, using a basic peptide by conjugating or mixing the RNA interfering agent with a basic peptide, e.g., a fragment of a TAT peptide, mixing with cationic lipids or formulating into particles.

Synthetic siRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for use in the methods and compositions of the disclosure. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective inhibitory molecules.

The term “subject” is intended to encompass a singular “subject” and plural “subjects” and includes, but is not limited to humans; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and bears.

The terms “inhibiting,” “reducing,” or “prevention,” or any variation of these terms, when used in the claims and/or the specification, includes any measurable decrease or complete inhibition to achieve a desired result. For example, there may be a decrease of 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity compared to normal. In a further example, following administering of an RNAi and/or NS1 protein inhibitor, a subject may experience a reduction in severity or duration of one or more viral infection symptoms.

The term “effective amount”, as used herein, refers to the amount that is safe and sufficient to treat, lesson the likelihood of, or delay the progress of a viral infection. The effective amount can thus cure or result in amelioration of the symptoms of the viral infection, slow the course of disease progression resulting from viral infection, slow or inhibit a symptom of a viral infection (e.g. flu symptoms), slow or inhibit the establishment of secondary symptoms of a viral infection or inhibit the development of a secondary symptom of a viral infection. The effective amount for the treatment of the viral infection depends on the type of viral infection to be treated, the severity of the symptoms, the subject being treated, the age and general condition of the subject, the mode of administration and so forth. Thus, it is not possible or prudent to specify an exact “effective amount”. However, for any given case, an appropriate “effective amount” can be determined by one of ordinary skill in the art using only routine experimentation. In one embodiment, the effective amount is a “therapeutically effective amount” for the alleviation of the symptoms of the disease mediated by the viral (e.g., influenza) infection or condition being treated. In another embodiment, the effective amount is a “prophylactically effective amount” for prophylaxis of the symptoms of the disease or condition being prevented.

For the purpose of the inhibition of influenza replication, the prophylaxis or treatment of influenza infection, the compositions of the disclosure can be administered by any means that produces contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds can, for example, be administered orally, transmucosally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically-acceptable carriers, adjuvants and vehicles. In certain embodiments, the compositions are administered as an oral preparation. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions of the disclosure and of ingredients suitable for use in said compositions is provided in Remington's Pharmaceutical Sciences, 18th edition, edited by A. R. Gennaro, Mack Publishing Co., 1990 and in Remington—The Science and Practice of Pharmacy, 21st edition, Lippincott Williams & Wilkins, 2005.

The terms “contacted” and “exposed,” when applied to a cell, are used herein to describe the process by which a composition of the disclosure is administered or delivered to a target cell or subject or are placed in direct juxtaposition with the target cell or subject. The terms “administered” and “delivered” are used interchangeably with “contacted” and “exposed.”

The methods of treatment according to the disclosure ameliorate one or more symptoms in a subject associated with the retroviral infection by preventing retroviral nucleic acid replication or decreasing the amount of retroviral nucleic acid replication in a subject, or preventing a productive infection. In some embodiments the retroviral infection is an influenza infection. The symptoms associated with influenza infection can include, but are not limited to, reduction in CD4+ T cell numbers, pain (peripheral neuropathy); fever, cough, and other cold/flu symptoms; night sweats; diarrhea, nausea, and other indigestion symptoms; lymph swelling or other immunological symptoms; weight loss and loss of appetite; candida in the mouth; secondary bacterial and/or viral infections; elevated liver enzymes; reduction in central nervous system and brain function; depression; overall reduced immunity; AIDS-related complications (ARC), including, but not limited to, progressive generalization lymphadenia (PGL), Kaposi's sarcoma, Pneumocystis carinii pneumonia, cataplectic purpura thrombocytopenica; neurological syndromes, including, but not limited to, dementia complications, encephalopathy, disseminated sclerosis ortropical paraplegia; as well as anti influenza antibody-positive and influenza-positive syndrome including that in silent patients.

“Treatment” and “treating” as used herein refer to administration or application of a therapeutic agent to a subject or performance of a procedure or modality on a subject for the purpose of obtaining a therapeutic benefit of a disease or health-related condition. For example, a subject or patient (e.g., a mammal, such as a human) having a viral infection may be subjected to a treatment comprising administration of a compound or composition of the disclosure.

The term “therapeutic benefit” or “therapeutically effective” as used throughout this application refers to anything that promotes or enhances the well-being of the subject with respect to the medical treatment of a condition. This includes, but is not limited to, a reduction in the onset, frequency, duration, or severity of the signs or symptoms of a disease (e.g., the flu). For example, a therapeutically effective amount of a compound or composition of the disclosure (e.g., an RNAi and/or NS1 protein inhibitor) may be an amount sufficient to treat or prevent a viral infection.

As used herein, the term “pharmaceutically acceptable”, and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. Each carrier must also be “acceptable” in the sense of being compatible with the other ingredients of the formulation. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. The pharmaceutical formulation contains a compound of the disclosure in combination with one or more pharmaceutically acceptable ingredients. The carrier can be in the form of a solid, semi-solid or liquid diluent, cream or a capsule. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the disclosure can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the disclosure that will be effective in the treatment of a particular disorder or condition will depend on the nature of the disorder or condition, and can be determined by standard clinical techniques. The phrase “pharmaceutically acceptable carrier or diluent” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body.

The following examples are intended to illustrate but not limit the disclosure. While they are typical of those that might be used, other procedures known to those skilled in the art may alternatively be used.

### EXAMPLES

Viruses and Cell Culture.

Human lung carcinoma cells (A549) were maintained in F-12K medium whereas human embryonic kidney cells (293T), Madin Darby canine kidney (MDCK) and African green monkey kidney epithelial cells (Vero) were cultured in Dulbecco's modified Eagle's medium (DMEM) containing 10% fetal bovine serum. Influenza A/WSN/1/33 (H1N1) (abbreviated here as WSN), and A/Puerto Rico/8/34 (H1N1) (abbreviated PR8-WT) and their NS1 deletions (abbreviated WSNΔNS1 and PR8ΔNS1, respectively) and Sendai virus were gifts. WSN and WSNΔNS1 were propagated respectively in MDCK cells and a MDCK cell line stably expressing NS1 fused with green fluorescence protein. As previously described (Li et al., 2010), virus titers and genotypes were further monitored by Western blot detection of the viral nucleocapsid protein (NP) and NS1. Nadomura virus (NoV) and its B2-deficient mutant (NoVΔB2) were rescued in BHK-21 cells (see, Li et al. Science, 342:231, 2013) from the infectious in vitro transcripts of full-length cDNA clones

Northern Blot Analysis.

A549 cells were seeded in a 10 cm plate at a density of 4×10⁶/plate one day before infection. Twelve hours after inoculation with serum-free DMEM (mock), WSN, WSNΔNS1 or Sendai viruses (moi=3), A549 cells were harvested for RNA extraction using TRIzol (Invitrogen, Carlsbad, Calif.) according to the manufacturer's protocol. ⅓ of the total yield of RNA from each sample was fractionated by denaturing polyacrylamide gel electrophoresis (PAGE) of either 6% or 15% for detecting the accumulation of pre- and mature miRNAs, respectively, and by 2% denaturing agarose gel electrophoresis for the analysis of pri-miRNAs. As described previously (Han et al., 2011), RNAs were electroblotted onto Hybond-NX membranes (Amersham Biosciences, Piscataway, N.J.) and chemically cross-linked (for pre- and mature miRNAs), or Hybond-N+ membranes (Amersham Biosciences, Piscataway, N.J.) and UV-cross-linked (for pri-miRNAs), and were hybridized with [γ-³²P]-ATP-labeled synthetic RNA oligonucleotide (Integrated DNA Technologies, San Diego, Calif.).

To determine the effect of NS1 on the cytoplasmic and nuclear accumulation of pre-miRNAs, 293T cells were seeded in a 6 cm plate at a density of 2×10⁶/plate and transfected with 4 μg of a pCMV-MIR expression plasmid using TransIT®-LT1 transfection reagent (Mirus, Madison, Wis.) following the supplier's recommended protocol. Human miRNA expression plasmids MIR-16, MIR-21, and MIR-100 were purchased from OriGene (Rockville, Md.) and each contained the pre-miRNA with 200-300 nucleotides up- and downstream flanking sequences amplified from human genomic DNA and cloned between the CMV promoter and a poly(A) tailing signal. Twelve hours after transfection, cells were infected by WSN or WSNΔNS1 (moi=3) and harvested 12 hours post infection in 1 mL 1×PBS by scraping and centrifugation at 300 g for 3 minutes. Cytoplasmic and nuclear fractions were prepared essentially as described (Hwang et al., 2007) with minor modifications. Cell pellets were resuspended by gentle pipetting in 800 μL lysis buffer A [10 mM Tris (pH 8.0), 140 mM NaCl, 1.5 mM MgCl₂, 0.5% Nonidet P-40] and incubated on ice for 5 minutes. After centrifugation at 1,000×g for 3 minutes at 4° C., an equal volume of phenol (Ambion, Austin, Tex.) was added to the supernatant for RNA purification, which contained the cytoplasmic fraction. Nuclei present in the pellets were washed twice with the lysis buffer A and resuspended in 1 mL Trizol for RNA extraction. Half of the total yield of RNA from total, nuclear and cytoplasmic fractions of each cell sample was used for Northern blot detection of pre- and mature miRNAs as described above. To ensure successful subcellular fractionation, the same filters were also probed for a mitochondria (mito) tRNA-Val and U6 RNA localized in the cytoplasm and nucleus, respectively (Hwang et al., 2007).

For Northern blot analysis of low molecular weight RNAs, 15 μg total RNA extracted from cells 24 hours after infection and a chemical cross-linking protocol were used. Instead of using locked nucleic acid (LNA) oligonucleotides as probes, the negative- and positive-strand influenza viral RNAs were detected by the ³²P-labeled synthetic RNA oligo, 5′-CAUAAUGGAUCCAAACACUGUG-3′ (SEQ ID NO:16) and 5′-GACACAGUGUUUGGAUCCAUUA-3′ (SEQ ID NO:17), respectively.

Plasmids and Molecular Cloning.

The coding sequences for the non-structural protein 1 (NS1) of PR8-WT and WSN-WT were obtained by RT-PCR from infected cells and cloned into pcDNA3.1 vector to generate pcDNA-PR8/NS1 and pcDNA-WSN/NS1. The expression plasmids in pCAGGS for VP35 of Ebola virus (EBOV), Marburg virus (MARV) and bat with an N-terminal Flag tag were kind gifts. The human Dicer expression plasmid was purchased from Addgene (no. 19873). The cDNA clones for Drosophila Loquacious isoforms PB and PD (Loqs-PB and Logs PD) were from Addgene (no. 41094 and 42095) whereas the plasmids encoding Drosophila proteins dDicer-1, dDicer-2, and R2D2 were gifts. The ORFs of Loqs-PB, Logs PD and R2D2 were cloned with an N-terminal Flag tag into pcDNAFlag to generate pcDNA-Flag-PB, pcDNA-Flag-PD and pcDNA-Flag-R2D2, respectively. The ORFs of dDicer-1 and dDicer-2 were cloned with an N-terminal His tag into pcDNA4HisMax to generate pcDNA-His-dDcr1 and pcDNA-His-dDcr2, respectively.

Suckling Mouse Infection and Construction of Small RNA Libraries.

BALB/c mice were purchased from Jackson labs (Bar Harbor, Me.). Animals were housed in the Animal Resources Facility according to the guidelines described under the Federal Animal Welfare Regulations Act. All animal procedures were approved by the Institutional Animal Care and Use Committee at the University of California, Riverside. NoV or NoVΔB2 preparation containing 7×10⁶ copies of genomic RNA1 from the titrated set of stocks was inoculated to each of suckling mice of 6 to 8 days old after birth by intraperitoneal injection (IP). Total RNAs were extracted from the hind limb tissues of mice 3 days post infection (dpi) with NoV, or 3, 7, 11, 15, 19, 23 and 27 dpi with NoVΔB2. These RNA preparations were used for the construction of small RNA libraries by the method that depends on the 5′ monophosphate of small RNAs as described previously (3) with the TruSeq Small RNA Sample Preparation Kit of Illumina (San Diego, Calif.). The same total RNA (1 μg) from the time course analysis of suckling mouse infection by NoVΔB2 was also used in quantitative RT-PCR analysis as described in Li et al. (Science 2013), to determine the copy number of the virus genomic RNA1 as a measurement of the in vivo virus titers.

Co-immunoprecipitation of small RNAs from mice 3 dpi with NoV or NoVΔB2 by Anti-pan Argonaute (Ago) antibody (Millipore, Billerica, Mass.), and from mice 3 dpi with NoV by B2 antibodies were as described in Li et al. (J. Biol. Chem. 2008). Briefly, 100 μg of muscle tissue lysates in 1 ml RIPA was pre-cleared by sequential incubation with 3 μg of rabbit or mouse IgG and 15 μl of protein A/G PLUS-Agarose beads (Santa Cruz Biotechnology, Santa Cruz, Calif.). Three μg of Anti-pan Ago or B2 antibodies immobilized to protein A/G PLUS-Agarose beads were then incubated with the pre-cleared cell lysates for 2 hours at 4° C. After extensive washes, the precipitated complexes were used for RNA extraction by TRIzol and the total RNAs obtained were used for the construction of small RNA libraries as described above.

Cell Culture Infection and Transfection and the Construction of Small RNA Libraries.

293T and hDcrKO 293T cells were seeded in a 6-cm plate at a density of 2.5×10⁶/plate one day before infection. Twenty-four hours after inoculation with serum-free DMEM (mock), PR8-WT, WSN-WT, PR8-ΔNS1 or WSN-ΔNS1 (moi=1) as previously described (Li et al., 2008), the infected cells were harvested for the extraction of total protein and RNA using TRIzol (Invitrogen, Carlsbad, Calif.) according to the manufacturer's protocol. To determine the role of Dicer in the biogenesis of viral siRNAs (vsiRNAs), hDicer-KO 293T cells seeded in a 6-cm plate at a density of 2.5×10⁶/plate were transfected with 8 μg of the plasmid encoding hDicer or dDicer-2, or co-transfected with (i) 8 μg pcDNA-His-dDcr1 and 4 μg pcDNA-Flag-PB, (ii) 8 μg pcDNA-His-dDcr2 and 4 μg pcDNA-Flag-PD, or (iii) 8 μg pcDNA-His-dDcr2, 4 μg pcDNA-Flag-PD, and 4 μg pcDNA-Flag-R2D2 using TransIT®-LT1 transfection reagent (Mirus, Madison, Wis.) following the supplier's recommended protocol. Six hours after transfection, hDcr-KO 293T cells were infected by PR8-ΔNS1 (moi=1) and the infected cells were harvested for the extraction of total protein and RNA using TRIzol 24 hours after infection. To determine the activity of viral suppressors of RNAi (VSRs), hDcr-KO 293T cells seeded in a 6-wells plate at a density of 1.3×10⁶/well were co-transfected with 4 μg of the hDicer expression plasmid with one (2 μg) of the following VSR-expressing plasmids: pcDNA-PR8/NS1, pcDNA-WSN/NS1, pCAGGS-EBOV/VP35, pCAGGS-MARV/VP35, or pCAGGS-bat/VP35. Six hours after co-transfection, the hDcr-KO 293T cells were infected by PR8 ΔNS1 (moi=1) and the infected cells were harvested for the extraction of total protein and RNA using TRIzol 24 hours after infection.

Libraries of small RNAs were constructed from total RNA extracted 24 hours after infection of 293T cells with PR8-ΔNS1 or WSN-ΔNS1 either without or with co-immunoprecipitation by Anti-pan Ago antibody (Millipore, Billerica, Mass.) as described above. Libraries of small RNAs were also constructed from (i) total RNA co-immunoprecipitated from 293T cells 24 hours after infection with PR8-WT or WSN-WT by Anti-pan Ago antibody (Millipore, Billerica, Mass.), (ii) total RNA from A549 cells 24 hours after infection with PR8-ΔNS1, (iii) total RNA from PR8-ΔNS1-infected hDcr-KO 293T cells ectopically expressing either hDicer or dDicer-2 together with Logs-PD and R2D2

Deep Sequencing and Bioinformatic Analysis of Small RNAs.

Libraries of small RNAs cloned from the hind limb tissues of suckling mice and cultured human cells were sequenced by Illumina HiSeq 2000/2500 at the Core Facility of the Institute for Integrative Genome Biology on campus. Small RNA reads were mapped to the virus and host genome references or compared to mature miRNAs. Mapping was done by Bowtie 0.12.9 with no mismatches. All of the references used were downloaded from web sources as listed below. Subsequent bioinformatics analysis of virus-derived small RNAs was carried out using in-house Perl scripts. Pairs of complementary 22-nt vsiRNAs in each library with different base-pairing lengths were computed using a previously described algorithm (Li et al., Science, 2013), which calculates the total counts of pairs in each nucleotide distance category between the 5′ and 3′ ends of complementary 22-nt vsiRNAs.

Semi-Quantitative RT-PCR.

A549 cells were harvested for RNA extraction with TRIzol. 12 hours post inoculation with serum-free DMEM (mock), WSN, WSNΔNS1 or Sendai viruses (moi=3). 1 μg total RNA from each sample was used as template for cDNAs synthesis using iScript™ Select cDNA Synthesis Kit (Bio-Rad, Richmond, Calif.). 1/200 of the cDNA products were used as template for PCR analysis using gene-specific primers as listed below and PCR products were resolved by 2% agarose gel electrophoresis and stained with ethidium bromide.

| Identifier | Sequence (SEQ ID NO:) |
|---|---|
| β-actin Fw | ACCAACTGGGACGACATGGAGAAA (18) |
| β-actin Bw | TAGCACAGCCTGGATAGCAACGTA (19) |
| IFNβ Fw | TGGGAGGCTTGAATACTGCCTCAA (20) |
| IFNβ Bw | TCTCATAGATGGTCAATGCGGCGT (21) |
| RIG-I Fw | AAACCAGAGGCAGAGGAAGAGCAA (22) |
| RIG-I Bw | TCGTCCCATGTCTGAAGGCGTAAA (23) |
| Pri-mir-27a fw | CCAGGGATTTCCAACCGACCC (24) |
| Pri-mir-27a bw | GCAGGATGGCAGGCAGACAGG (25) |
| Pri-mir-100 fw | AGACATGTCACAGCCCCAAAAGAGAG (26) |
| Pri-mir-100 bw | AAGGAAACTAAGGGGAAGAGAAGGAG (27) |
| pri-mir-let-7a- | GATTCCTTTTCACCATTCACCCTGGATGTT (28) |
| 1 fw | |
| pri-mir-let-7a- | TTTCTATCAGACCGCCTGGATGCAGACTTT (29) |
| 1 bw | |
| pri-miR-21 fw | GTTCGATCTTAACAGGCCAGAAATGCCTGG (30) |
| pri-miR-21 bw | ACCAGACAGAAGGACCAGAGTTTCTGATTA (31) |
| pri-mir-16-1 fw | GAAAAGGTGCAGGCCATATTGT (32) |
| pri-mir-16-1 bw | CGCCAATATTTACGTGCTGCTA (33) |

Western Blot Analysis.

Western blot analysis was performed as described previously with minor modifications (Li et al., J. Biol. Chem 283:23397, 2008). Antibodies to NS1 and NP were gifts of Dr. Yan Zhou and those to hDicer and PACT (Santa Cruz Biotechnology, Santa Cruz, Calif.), Ago2 (Active Motif LLC, Carlsbad, Calif., USA), TRBP (Abnova, Taipei, Taiwan), Ago1 and β-Actin (Cell Signaling Technology, Beverly, Mass.) were from commercial suppliers. To determine the effect of NS1 on the cytoplasmic and nuclear accumulation of RISC components, 293T cells were inoculated and harvested for preparing total, cytoplasmic and nuclear fractions as described above. 1/50 of the supernatant from each sample was directly mixed with protein loading buffer whereas the nuclei pellets after washing were used for both protein and RNA extraction with Trizol following the supplier's recommended protocol and 1/20 of the nuclear proteins from each sample was used for Western blot analysis. Following SDS PAGE and transfer to nitrocellulose membranes (Bio-Rad, Richmond, Calif.), membranes were probed overnight at 4° C. with primary antibodies after blocking with Tris-buffered saline containing 0.1% Tween-20 and 5% skim milk for 1 hour at room temperature. NS1 was detected by alkaline phosphatase-conjugated anti-rabbit IgG (Anaspec, San Jose, Calif.) with BCIB/NBT premix solution (Sigma-Aldrich, St Louis, Mo.). For the detection of the less abundant cellular proteins, HRP-conjugated anti-rabbit or anti-mouse IgG secondary antibodies (Thermo Fisher Scientific, Rockford, Ill.) were used with an enhanced chemiluminescence reagent (Amersham Biosciences, Piscataway, N.J.).

Analysis of RNA-Protein Interactions.

The protocol described by Chi et al., 2009 was used for UV cross-linking of protein-RNA in living cells with modifications. A549 and 293T cells 12 hours post infection with WSN were washed twice with PBS, placed in XL-1000 UV crosslinker (Spectronics corporation, Rolling Meadows, Ill.) with the cover off, irradiated once for 400 mJ/cm² and another for 200 mJ/cm². Cells were then collected and lysates prepared by the commercial cell lysis buffer (Cell Signaling Technology, Beverly, Mass.) for SDS PAGE and Western blot analysis using NS1 antibodies as described above. In contrast to the detection of NS1 by alkaline phosphatase-conjugated secondary antibodies, NS1 complexes were visualized by chemiluminescence.

Co-immunoprecipitation of NS1-pre-miR16 complex by NS1 antibodies was essentially as described (Li et al., 2008). Briefly, lysates of 293T cells transfected with pCMV-MIR-16 and subsequently inoculated with serum-free DME (mock), WSN or WSNΔNS1 were prepared as described above. 600 μl of whole cell lysates was pre-cleared by sequential incubation with 3 μg of rabbit IgG and 15 μl of protein A/G PLUS-Agarose beads (Santa Cruz Biotechnology, Santa Cruz, Calif.). Three μg of NS1 antibodies immobilized to protein A/G PLUS-Agarose beads were then incubated with the precleared cell lysates for 2 hours at 4° C. After extensive washes, the precipitated NS1 complexes were used for RNA extraction by TRIzol and the total RNA molecules obtained were analyzed by Northern blot hybridizations to detect pre-miR-16 and U6 snRNA.

Purification of Strep-tagged NS1 protein complexes was achieved using a recombinant Influenza A/Puerto Rico/8/34 (H1N1) (abbreviated here as PR8) essentially as described (Lin et al., 2012). An eight-plasmid reverse genetics system (Quinlivan et al., 2005) for PR8, a gift of Dr. Peter Palese, was used to generate the recombinant PR8 virus. Strep-Tag II (WSHPQFEK; (SEQ ID NO:35))-encoding sequence (TGGTCACACCCACAGTTCGAAAAA; (SEQ ID NO:34)) was introduced into PR8 NS segment with the tag inserted after amino acid 79 by standard overlapping PCR (Li et al., 2010). The mutant virus, Strep rPR8, was rescued by co-transfection of a mixture of 293T and MDCK cells with the pDZ-PB2, pDZ-PB1, pDZ-PA, pDZ-HA, pDZ-NP, pDZ-NA, and pDZ-M plus the modified pDZ-NS. As found previously (Lin et al., 2012), Strep rPR8 had similar growth properties to wild-type PR8 virus during single-cycle replication. 293T cells were infected with Strep rPR8 at MOI of 3. At 12 hour post infection, cells were harvested and lysed in the commercial cell lysis buffer. After sonication, cell lysates were clarified by centrifugation at 12,000×g for 5 min at 4° C., and the supernatant was incubated with Strep-Tactin Sepharose (IBA, Göttingen, Germany) for 2 hours at 4° C. The Sepharose beads were washed with washing buffer (100 mM Tris-Cl, pH 8.0, 150 mM NaCl, 1 mM EDTA) for 5 times and the bound proteins eluted with elution buffer (washing buffer plus 2.5 mM desthiobiotin). RNA in the pulled down NS1 complexes was extracted by TRIzol and analyzed by Northern blot hybridizations with probes specific to individual pri-, pre- and mature miRNAs.

The total RNA extracted from the NS1 complexes pulled down by Strep-Tactin Sepharose was also labeled at the 3′-ends. The labeling reaction was carried out at 4° C. overnight in an RNase-free microfuge tube containing 50-100 pmol of the extracted RNA, 50-100 pmol [³²P]pCp and 10 U T4 RNA Ligase (New England Biolabs, Beverly, Mass.) in a total reaction volume of 20 μl. Unincorporated [³²P]pCp was removed by applying the mixture to an RNase-free MicroSpin™ G-50 spin column (Amersham Biosciences, Buckinghamshire, United Kingdom) following the manufacturer's recommendations. The [32P]pCp-labeled RNA was fractionated by 15% denaturing PAGE and the gel exposed to Molecular Imager FX (Bio-Rad, Richmond, Calif.). The pulled down RNA and the Low Range ssRNA Ladder (New England Biolabs, Beverly, Mass.) were also labeled with RNA 3′ End Biotinylation Kit (Thermo Fisher Scientific, Rockford, Ill.) according to the manufacturer's instructions and fractionated by 6% denaturing PAGE. The experiments in this study were repeated three times.

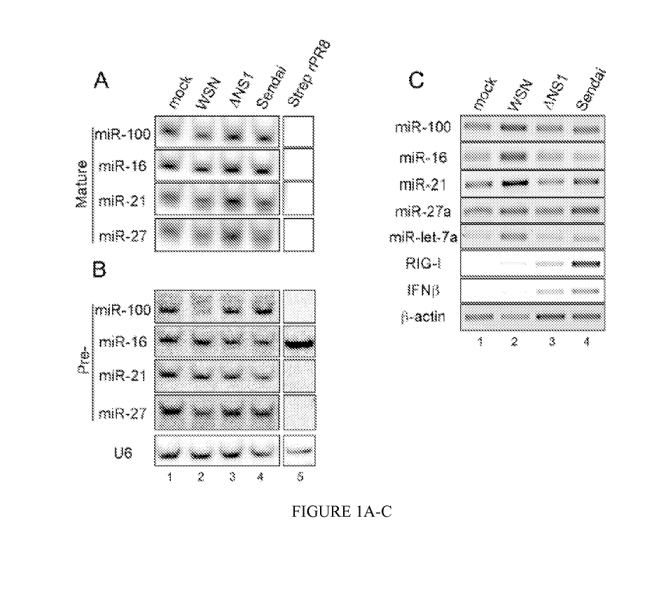

Northern blot hybridization was used to verify changes in the accumulation of specific host miRNAs correlated with NS1 expression in human lung epithelial cells (A549) infected with strain A/WSN/33 (WSN) that were identified initially by deep sequencing of total small RNAs. The accumulation of miR-16 and miR-100, but not miR-21 or miR-27, was reproducibly decreased by 25-35% in A549 cells after wildtype WSN infection as compared to mock-inoculated cells or cells infected with the NS1-deficient mutant virus (WSNΔNS1) or Sendai virus (FIG. 1A). Notably, unlike infection with either WSNΔNS1 or Sendai virus, precursor miR-100 (pre-miR-100) became hardly detectable in WSN-infected cells (FIG. 1B). In contrast to the suppression of pre-miR-100, detectable also at 24 hours post infection (FIG. 5), accumulation of pre-miR-16, pre-miR-21 and miR-27 was not obviously altered following WSN infection (FIG. 1B). These findings suggest that expression of NS1 during influenza viral infection interferes with biogenesis of miR-16 and miR-100 at distinct steps.

Mammalian miRNAs are cleaved by Dicer in the cytoplasm from ˜70-nucleotide stem-loop pre-miRNAs that are processed from primary miRNA transcripts (pri-miRNAs) in the nucleus and subsequently exported. Therefore, the accumulation of pri-miRNAs in A549 cells was compared before and after infection by reverse transcription-polymerase chain reaction (RT-PCR). As expected, NS1-mediated suppression of transcription of RIG-I and IFN-β was detected, which are innate immune pathway genes induced in cells by Sendai or WSNΔNS1 challenges (FIG. 1C). However, NS1 exhibited no inhibitory effect on the accumulation of either pri-miR-16 or pri-miR-100 (FIG. 1C). All of the five examined pri-miRNAs accumulated to similar or higher levels in A549 cells after infection with WSN as compared to infection with WSNΔNS1 or Sendai virus (FIG. 1C). Together with the findings described above, these results suggest that NS1 inhibits the nuclear production or stability of pre-miR-100 whereas NS1 may target a step further downstream in the biogenesis of miR-16.

NS1 is a dsRNA-binding protein accumulating predominantly in the nucleus of the infected cells. It was thus hypothesized that NS1 inhibition was related to the biogenesis of specific miRNAs might also involve direct interactions with the structured miRNA precursors. Experiments with protein-RNA cross-linking by UV treatment indeed led to formation of high molecular weight complexes that migrated much slower than free NS1 of ˜27 kD in WSN-infected A549 cells (FIG. 2A). By comparison, these NS1-RNA complexes were more readily detectable in human embryonic kidney 293T cells than in A549 cells (FIG. 2A). Co-immunoprecipitation with NS1 antibodies further indicated that NS1 and pre-miR-16 were present in a complex in the infected cells (FIG. 2B). U6 small nuclear RNA (snRNA) was also co-immunoprecipitated by NS1 antibodies, consistent with a previous study.

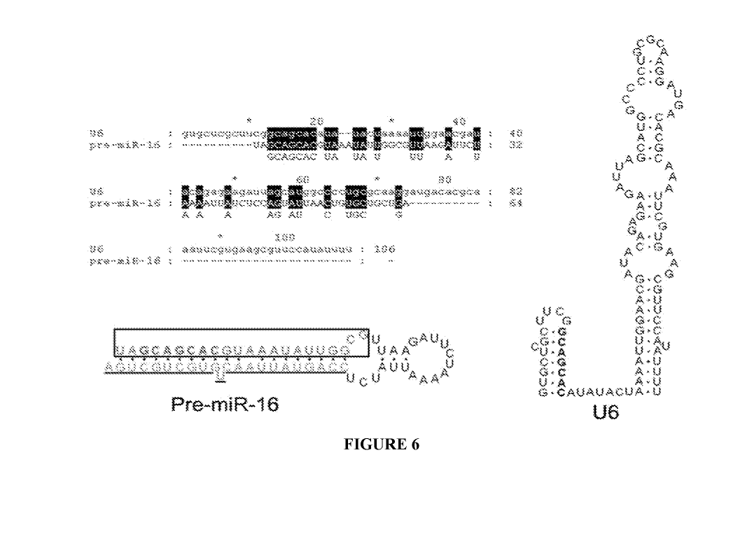

The observed NS1-RNA interactions were further investigated using a recombinant Influenza A virus expressing NS1 with an inserted 8-amino acid Strep-Tag II, which has binding specificity comparable to biotin, thereby allowing high affinity purification of NS1 complexes formed during infection. The low and high molecular weight RNAs associated with NS1 either by Northern blot hybridization were examined after fractionation in polyacrylamide and agarose gels or by end-labeling. The data showed that both pre-miR-16 and U6 snRNA were specifically pulled down together with NS1 from the infected 293T cell lysates (FIGS. 1A/1B, lane 5; FIG. 2C). miR-100-specific RNA molecules of ˜1.5 to 2 kilobases were also detected from the pulled down RNAs (FIG. 2D, right panel), which likely corresponded to pri-miR-100. However, Northern blot hybridizations failed to detect pre-miR-21, pre-miR-100, pre-miR-27 (FIG. 1B, lane 5; FIG. 2C), or any mature miRNAs (FIG. 1A) in the pulled down NS1 complexes. Binding of NS1 to RNAs in the size ranges of pre-miRNAs and U6 snRNA, but not of mature miRNAs, was further verified by end-labeling of total RNAs pulled down with NS1 by Strep-Tactin Sepharose beads (FIG. 2E). Interestingly, sequence similarity was shared between U6 snRNA and pre-miR-16 in the region coding for mature miR16 (FIG. 6). These results together demonstrate direct interactions of NS1 with endogenous pre-miR-16 and pri-miR-100 in addition to U6 snRNA in the infected human cells.

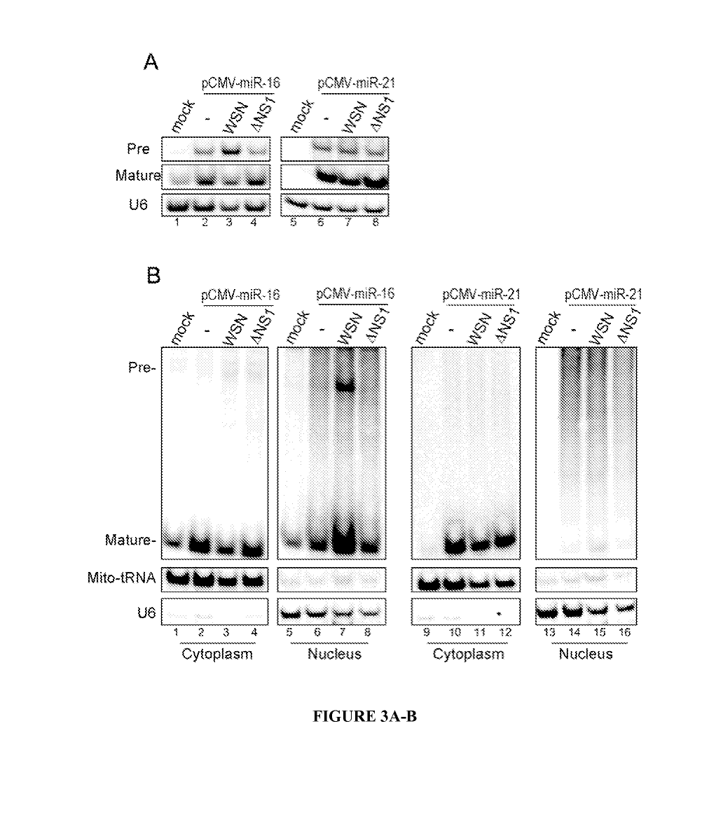

These findings were notable since they provided the first evidence for direct interaction of a mammalian viral protein with specific host miRNA precursors during infection. Direct NS1 binding to pri-miR-100, possibly to a specific structural element outside pre-miR-100, may inhibit the processing of pri-miR-100 into pre-miR-100, resulting in the observed reduction in the accumulation of both pre-miR-100 and mature miR-100 (FIGS. 1A/1B). It was hypothesized that direct NS1 binding to pre-miR-16 might prevent the nuclear export of pre-miR-16. To test the hypothesis, miRNA precursors were over-expressed in 293T cells by transfection with commercial miRNA-expressing plasmids to allow easy detection of pre-miRNAs. Under these conditions, NS1 expression was also associated with significantly reduced accumulation of mature miR-16 in the infected cells (FIG. 3A). RNAs from the cytoplasmic and nuclear extracts was isolated 12 hours after infection with either WSN or WSNΔNS1 for RNA gel blot analysis to probe miRNAs, tRNA (a cytoplasmic RNA), and U6 snRNA (a nuclear RNA). The results showed that pre-miR-16, but not pre-miR-21, accumulated to high levels in the nucleus of cells infected with wild type WSN as compared to that in cells either mock-inoculated or infected with WSNΔNS1 (FIG. 3B). Consistently, the accumulation of the cytoplasmic mature miR-16 was lower in WSN-infected cells than in cells either mock-inoculated or infected with WSNΔNS1 (FIG. 3B). These findings indicate that NS1 expression is indeed associated with specific nuclear retention of pre-miR-16 in the infected cells, thereby restricting Dicer access to pre-miR-16 and reducing the production of mature miR-16 in the cytoplasm.

MiR-16 is among a few human miRNAs that also accumulate to detectable levels in the nucleus. Transfection with pCMV-miR-16 further enhanced nuclear accumulation of miR-16 (FIG. 3B, lanes 5 and 6). Interestingly, as was found for pre-miR-16, mature miR-16 also accumulated to much higher levels in the nucleus of cells infected with wild type WSN than cells either mock-inoculated or infected with WSNΔNS1 (FIG. 3B, compare lane 7 with lanes 6 and 8). A modest but reproducible increase in the nucleus was also observed for miR-21 (FIG. 3B, compare lane 15 with lanes 14 and 16), which, like most human miRNAs, was undetectable in the nucleus without transfection with pCMV-miR-21 (FIG. 3B). Thus, NS1 expression appears to facilitate nuclear sequestration of host miRNAs in a non-selective manner during infection.

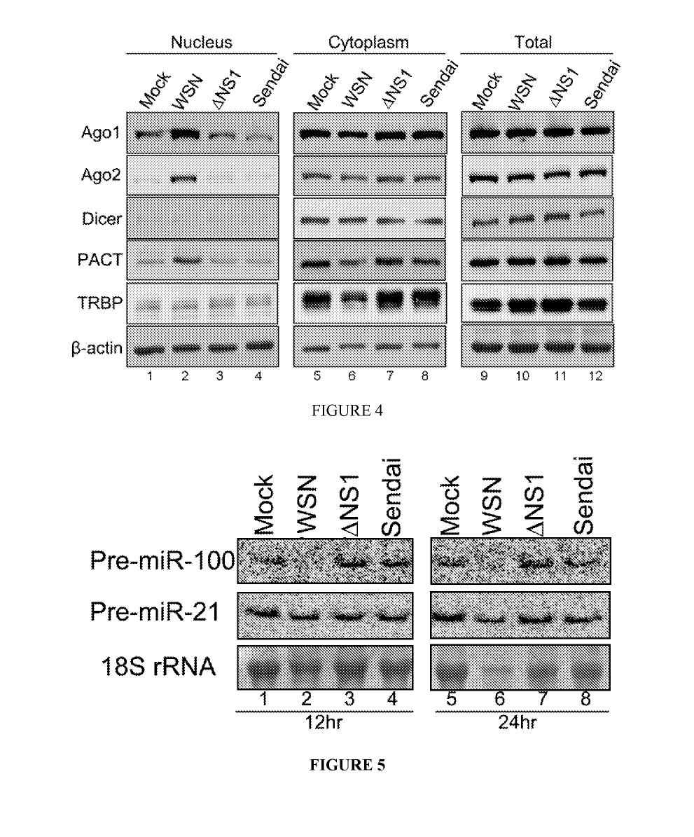

Human miRNAs are eventually loaded into one of the four AGOs in an RNA-induced silencing complex (RISC) by RISC-loading complex consisting of Dicer, AGO and one or both of the dsRNA binding proteins, TRBP and PACT. Western blot analysis did not reveal any major differences in the distribution of either Dicer or TRBP between the nucleus and cytoplasm in 293T cells before and after infection with WSN or WSNΔNS1 (FIG. 4). In contrast, infection of 293T cells with WSN, but not WSNΔNS1, resulted in a significantly enhanced accumulation of AGO1, AGO2 and PACT in the cell nucleus (FIG. 4). These results indicate that NS1 expression induces nuclear translocation of specific components of the host RISC, which is likely to compromise the function of many miRNAs. Translocation of AGO1 or AGO2 loaded with mature miRNAs into the nucleus may explain the observed nuclear sequestration of host miRNAs in NS1-expressing wild type virus-infected cells, possibly in a manner similar to AGO-mediated nuclear import of endogenous siRNAs.

The results identify the structured precursors of host miR-16 and miR-100 as the endogenous RNA targets of the influenza viral dsRNA-binding protein NS1. Among the validated targets of miR-100, mTOR is a key component of the phosphatidylinositol-3-kinase (PI3K) pathway, activation of which promotes infection of many viruses including Influenza A virus. This suggests that NS1-mediated mTOR de-repression by blocking miR-100 function may contribute to the recently described activation of PI3K signaling by NS1. Accordingly, in one embodiment of the disclosure inhibition of mTOR and PI3K pathways is provided, which inhibits influenza infection. In one embodiment, the mTOR/PI3K pathway is inhibited by using exogenous miR-100. Notably, miR-16 induces apoptosis by repressing the expression of Bcl-2, which suppresses apoptosis. Therefore, removal of Bcl-2 repression by NS1 suppression of miR-16 function provides a new insight into the mechanism of suppression of apoptosis by NS1 in the infected human cells. Accordingly the disclosure also provide a method of inhibiting influenza infection by inhibiting Bcl-2 expression. In one embodiment, Bcl-2 expression is inhibited by miR-16. These observations together indicate that suppression of cellular miRNA function by NS1 may have a unique pathogenic role and contribute to the observed suppression of the host innate immune responses during single and mixed influenza A virus infections.

Several DNA viruses encode miRNAs to influence viral pathogenesis while other viruses may activate or repress expression of specific cellular miRNAs during infection. The foregoing data provides the first example for specific inhibition of cellular miRNA biogenesis by a mammalian viral protein. NS1-mediated depletion of pre-miR-100 by direct binding to pri-miR-100 appears mechanistically similar to Lin28B, a mammalian RNA-binding protein that sequesters pri-let-7 and inhibits its processing into pre-let-7 in the nucleus. However, specific binding and nuclear sequestration of pre-miR-16 by NS1 provides a new mechanism to regulate the function of miRNAs. This mechanism is distinct to the specific inhibition of miRNA biogenesis by adeno- and herpesviral noncoding RNAs. Adenoviral VA1 RNA appears to saturate the function of Exportin 5 to inhibit nuclear export of pre-miRNAs whereas herpesviral HSUR 1 directly binds to and depletes mature miR-27. A number of mammalian viruses encode proteins capable of suppressing experimentally induced RNAi mediated by siRNAs. These include VP35, E3L and NSs encoded by Ebola, Vaccinia and La Crosse viruses that bind to dsRNA in vitro but share no significant sequence similarities to NS1. However, a physiological role for the RNAi suppression activity of these proteins during infection was unclear and conflicting results were reported in the literature on the RNAi suppression activity of some of these proteins in mammalian cells. Suppression of the function of specific host miRNAs by NS1 under physiological conditions demonstrated in this work therefore suggests a new paradigm for the action of mammalian viral suppressors of RNAi.

The reference sequences used in this study:
Nodamura Virus (NoV) RNAs 1 and 2: NCBI NC_002690.1 and NC_002691.1.
NoVΔB2 RNAs 1 and 2: the same as NoV except for 3 substitutions in RNA1: U2745C, U2754C and C2757G.
A/Puerto Rico/8/34 (H1N1) (PR8-WT): NCBI AF389115.1, AF389116.1, AF389117.1, AF389118.1, AF389119.1, AF389120.1, AF389121.1 and AF389122.1.
PR8-ΔNS1: the same as PR8-WT except for the deletion of nucleotides 57 to 528 in NS segment.
A/WSN/1/33 (H1N1) (WSN-WT): NCBI J02179.1, J02178.1, CY034137.1, J02176.1, CY034135.1, L25817.1, L25818.1 and M12597.1 with the missing terminal sequences from the following segments completed by sequencing (nucleotides underlined are the original terminals from NCBI):
Segment PA (segment 3) 3′ terminal:

(SEQ ID NO: 36)
AGCGAAAGCAGGTACTGATT . . .

Segment PA (segment 3) 5′ terminal:

(SEQ ID NO: 37)
. . . CAAAAAAGTACCTTGTTTCTACT

Segment NP (segment 5) 3′ terminal:

(SEQ ID NO: 38)
AGCAAAAGCAGGGTAGATAATCACTC . . .

Segment NP (segment 5) 5′ terminal:

(SEQ ID NO: 39)
. . . AAAGAAAAATACCCTTGTTTCTACT

Segment M (segment 7) 5′ terminal:

(SEQ ID NO: 40)
. . . AAACTACCTTGTTTCTACT

WSN-ΔNS1: the same as WSN-WT except for the deletion of nucleotides 57 to 528 in NS segment.
Mature miRNAs and miRNA precursors: miRBase 19.
Mus musculus non-coding RNAs: fRNAdb 3.0.
Mus musculus mRNAs: Mammalian Gene Collection (MGC) and NIA Mouse cDNA Project.
Mus musculus whole genome: the December 2011 (GRCm38/mm10) assembly of the mouse genome (mm10, Genome Reference Consortium Mouse Build 38 (GCA_000001635.2)).

A number of embodiments of the disclosure have been described. Nevertheless, it will be understood that various modifications may be made without departing from the spirit and scope of the disclosure. Accordingly, other embodiments are within the scope of the following claims.

1. (canceled)

2. A method of treating an influenza infection comprising inhibiting mTOR/PI3K pathway activation.

3. The method of claim 2, wherein the inhibiting is by promoting expression or the amount of an RNAi molecule comprising an miR-100 sequence in a cell or subject.

4. The method of claim 2, wherein the RNAi molecule comprising an miR-100 sequence is delivered to the cell or subject.

5. The method of claim 4, wherein the delivery is by intranasal or intraocular administration to a subject.

6. The method of claim 3, wherein the RNAi molecule comprising an miR-100 sequence is delivered in a pri-miR-100 form.

7. A method of treating an influenza infection comprising inhibiting Bcl-2 activation or expression.

8. The method of claim 7, wherein the inhibiting is by promoting expression or the amount of an RNAi molecule comprising an miR-16 sequence in a cell or subject.

9. The method of claim 7, wherein the RNAi molecule comprising an miR-16 sequence is delivered to the cell or subject.

10. The method of claim 9, wherein the delivery is by intranasal or intraocular administration to a subject.

11. The method of claim 8, wherein the RNAi molecule comprising an miR-16 sequence is delivered in a pri-miR-16 form.

12. A method for treating an influenza viral infection comprising administering a composition comprising (i) an RNAi comprising a miR-100 sequence; (ii) an RNAi comprising a miR-16 sequence, (iii) an NS1 protein inhibitor or (iv) any combination of (i), (ii), and (iii).

13-16. (canceled)


SEQUENCE LISTING (41 sequences)

SEQ ID NO: 1 - RNA, 22 nt, Homo sapiens
uagcagcacguaaauauuggcg

SEQ ID NO: 2 - RNA, 89 nt, Homo sapiens
gucagcagugccuuagcagcacguaaauauuggcguuaagauucuaaaauuaucuccaguauuaacugugcugcugaaguaagguugac

SEQ ID NO: 3 - RNA, 81 nt, Homo sapiens
guuccacucuagcagcacguaaauauuggcguagugaaauauauauuaaacaccaauauuacugugcugcuuuagugugac

SEQ ID NO: 4 - RNA, 22 nt, Homo sapiens
aacccguagauccgaacuugug

SEQ ID NO: 5 - RNA, 80 nt, Homo sapiens
ccuguugccacaaacccguagauccgaacuugugguauuaguccgcacaagcuuguaucuauagguaugugucuguuagg

SEQ ID NO: 6 - RNA, 88 nt, Artificial Sequence
  vsiRNA sequence reads
cgaaagcaggucaauuauauucaauauggaaagaauaaaagaacuaagaaaucuaaugucgcagucucgcacccgcgagauacucaca

SEQ ID NO: 7 - RNA, 22 nt, Artificial Sequence
  vsiRNA sequence reads
ucgcuuucguccaguuaauaua

SEQ ID NO: 8 - RNA, 23 nt, Artificial Sequence
  vsiRNA sequence reads
ugauucuuuagauuacagcguca

SEQ ID NO: 9 - RNA, 86 nt, Artificial Sequence
  vsiRNA sequence reads
caaaagcagggugacaaagacauaauggauccaaacacugugucaagcuuucaggacauacugcugaggaugucaaaaaugcaguu

SEQ ID NO: 10 - RNA, 86 nt, Artificial Sequence
  vsiRNA sequence reads
ucguuuucgucccacuguuucuguauuaccuagguuugugacacaguucgaaaguccuguaugacgacuccuacaguuuuuacguc

SEQ ID NO: 11 - RNA, 87 nt, Artificial Sequence
  vsiRNA sequence reads
cgaaagcaggucaauuauauucaauauggaaagaauaaaagaacuaaggaaucuaaugucgcagucucgcacucgcgagauacucac

SEQ ID NO: 12 - RNA, 22 nt, Artificial Sequence
  vsiRNA sequence reads
ucgcuuucguccaguuaauaua

SEQ ID NO: 13 - RNA, 23 nt, Artificial Sequence
  vsiRNA sequence reads
ugauuccuuagauuacagcguca

SEQ ID NO: 14 - RNA, 87 nt, Artificial Sequence
  vsiRNA sequence reads
caaaagcagggugacaaagacauaauggauccaaacacugugucaagcuuucaggacauacugaugaggaugucaaaaaugcaguug

SEQ ID NO: 15 - RNA, 86 nt, Artificial Sequence
  vsiRNA sequence reads
ucguuuucgucccacuguuucuguauuaccuagguuugugacacaguucgaaaguccuguaugacuacuccuacaguuuuuacguc

SEQ ID NO: 16 - RNA, 22 nt, Artificial Sequence
  RNA oligo probe
cauaauggauccaaacacugug

SEQ ID NO: 17 - RNA, 22 nt, Artificial Sequence
  RNA oligo probe
gacacaguguuuggauccauua

SEQ ID NO: 18 - DNA, 24 bp, Artificial Sequence
  Beta-actin forward primer
accaactgggacgacatggagaaa

SEQ ID NO: 19 - DNA, 24 bp, Artificial Sequence
  Beta-actin reverse primer
tagcacagcctggatagcaacgta

SEQ ID NO: 20 - DNA, 24 bp, Artificial Sequence
  IFN-beta forward primer
tgggaggcttgaatactgcctcaa

SEQ ID NO: 21 - DNA, 24 bp, Artificial Sequence
  IFN-beta reverse primer
tctcatagatggtcaatgcggcgt

SEQ ID NO: 22 - DNA, 24 bp, Artificial Sequence
  RIG-1 forward primer
aaaccagaggcagaggaagagcaa

SEQ ID NO: 23 - DNA, 24 bp, Artificial Sequence
  RIG-1 reverse primer
tcgtcccatgtctgaaggcgtaaa

SEQ ID NO: 24 - DNA, 21 bp, Artificial Sequence
  Pri-MIR-27a forward primer
ccagggatttccaaccgaccc

SEQ ID NO: 25 - DNA, 21 bp, Artificial Sequence
  Pri-MIR-27a reverse primer
gcaggatggcaggcagacagg

SEQ ID NO: 26 - DNA, 26 bp, Artificial Sequence
  Pri-MIR-100 forward primer
agacatgtcacagccccaaaagagag

SEQ ID NO: 27 - DNA, 26 bp, Artificial Sequence
  Pri-MIR-100 reverse primer
aaggaaactaaggggaagagaaggag

SEQ ID NO: 28 - DNA, 30 bp, Artificial Sequence
  Pri-MIR-let-7a-1 forward primer
gattccttttcaccattcaccctggatgtt

SEQ ID NO: 29 - DNA, 30 bp, Artificial Sequence
  Pri-MIR-let-7a-1 reverse primer
tttctatcagaccgcctggatgcagacttt

SEQ ID NO: 30 - DNA, 30 bp, Artificial Sequence
  Pri-MIR-21 forward primer
gttcgatcttaacaggccagaaatgcctgg

SEQ ID NO: 31 - DNA, 30 bp, Artificial Sequence
  Pri-MIR-21 reverse primer
accagacagaaggaccagagtttctgatta

SEQ ID NO: 32 - DNA, 22 bp, Artificial Sequence
  Pri-MIR-16-1 forward primer
gaaaaggtgcaggccatattgt

SEQ ID NO: 33 - DNA, 22 bp, Artificial Sequence
  Pri-MIR-16-1 reverse primer
cgccaatatttacgtgctgcta

SEQ ID NO: 34 - DNA, 24 bp, Artificial Sequence
  Strep-Tag II
tggtcacacccacagttcgaaaaa

SEQ ID NO: 35 - protein, 8 aa, Artificial Sequence
  Synthetic Construct
WSHPQFEK

SEQ ID NO: 36 - DNA, 20 bp, Artificial Sequence
  Segment PA
agcgaaagcaggtactgatt

SEQ ID NO: 37 - DNA, 23 bp, Artificial Sequence
  Segment PA
caaaaaagtaccttgtttctact

SEQ ID NO: 38 - DNA, 26 bp, Artificial Sequence
  Segment NP
agcaaaagcagggtagataatcactc

SEQ ID NO: 39 - DNA, 25 bp, Artificial Sequence
  Segment NP
aaagaaaaatacccttgtttctact

SEQ ID NO: 40 - DNA, 19 bp, Artificial Sequence
  Segment M
aaactaccttgtttctact

SEQ ID NO: 41 - RNA, 106 nt, Artificial Sequence
  U6 promoter sequence
gugcucgcuucggcagcacauauacuaaaauuggaacgauacagagaagauuagcauggccccugcgcaaggaugacacgcaaauucgugaagcguuccauauuuu